(19) United States
(12) Patent Application Publication
(10) Pub. No.: US 2007/0259962 A1
(43) Pub. Date: Nov. 8, 2007

(54) Use of N-Arylhydrazine Derivatives for Combating Pests in and on Animals

(76) Inventors: Wolfgang von Deyn, Neustadt (DE); Hassan Oloumi-Sadeghi, Raleigh, NC (US); David G. Kuhn, Apex, NC (US); Nigel Armes, Raleigh, NC (US); Christopher Koradin, Stadtallendorf (DE); Alissa Zeller, Mannheim (DE)

Correspondence Address: Hutchison Law Group, P.O. Box 31686, Raleigh, NC, 27612, US

(21) Appl. No.: 10/580,700

(22) PCT Filed: Dec. 2, 2004
(86) PCT No.: PCT/EP04/13685

§ 371 Date: Mar. 15, 2007

Related U.S. Application Data
(60) Provisional application No. 60/526,609, filed on Dec. 4, 2003.

(51) Int. Cl. A61K 31/155; A61P 33/00

(52) U.S. Cl. 514/634

(57) ABSTRACT

Use of compounds of formula I 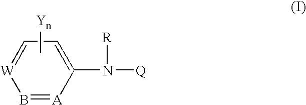
wherein Q is 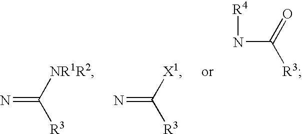
X¹ is chlorine, bromine, or fluorine;
R¹, R² are each independently H, alkyl, alkenyl, alkynyl, or cycloalkyl, alkylamino, dialkylamino, alkylcarbonylamino, alkylsulfonyl, or alkylsulfinyl, wherein the carbon atoms in these groups may be substituted, or
R¹ and R² may be taken together to form a ring represented by the structure 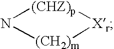 p, m are 1, 2 or 3; X′ is oxygen, sulfur, amino, alkylamino, phenylamino, or methylene; Z is alkyl or phenyl;
R³ is H, alkyl, alkenyl, alkynyl, cycloalkyl, wherein the carbon atoms in these groups may be substituted;
R, R⁴ are H or alkyl, alkoxycarbonyl, alkylaminocarbonyl, or dialkylaminocarbonyl, wherein the carbon atoms in the these groups may be substituted;
A is C—R⁵ or N; B is C—R⁶ or N; W is C—R⁷ or N; with the proviso that one of A, B and W is other than N;
R⁵, R⁶, R⁷ are H, halogen, nitro, cyano, amino, mercapto, hydroxy, alkyl, alkenyl, alkynyl, cycloalkyl, alkoxy, alkylamino, dialkylamino, alkylthio, alkylsulfonyl, or alkylsulfinyl, wherein the carbon atoms in these groups may be substituted, a 5 to 6-membered aromatic ringsystem which may contain 1 to 4 heteroatoms selected from oxygen, sulfur and nitrogen and which may be substituted; Y is hydrogen, halogen, cyano, nitro, amino, hydroxy, mercapto, alkyl, alkenyl, alkynyl, cycloalkyl, alkoxy, alkylamino, dialkylamino, alkylthio, alkylsulfonyl, or alkylsulfinyl, wherein the carbon atoms in these groups may be substituted; n is 0, 1, or 2; for combating parasites in and on animals.

The present invention relates to the use of hydrazine derivatives of formula I: 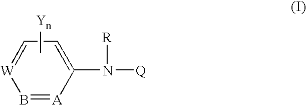
wherein
Q is 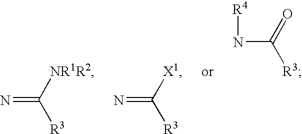
X¹ is chlorine, bromine, or fluorine;
R¹, R² are each independently hydrogen, C₁-C₁₀-alkyl, C₃-C₁₀-alkenyl, C₃-C₁₀-alkynyl, or C₃-C₁₂-cycloalkyl, C₁-C₆-alkylamino, di(C₁-C₆-alkyl)-amino, C₁-C₆-alkylcarbonylamino, C₁-C₆-alkylsulfonyl, or C₁-C₆-alkylsulfinyl, wherein the carbon atoms in these groups may be substituted with
   1 to 3 halogen, hydroxy, nitro, cyano, amino, mercapto, C₁-C₆-alkoxy, C₁-C₆-haloalkoxy, C₁-C₆-alkylthio, C₁-C₆-haloalkylthio, C₁-C₆-alkylsulfonyl, C₁-C₆-alkylsulfinyl, C₁-C₆-haloalkylsulfonyl, C₁-C₆-haloalkylsulfinyl, or C₃-C₆-cycloalkyl which may be substituted with 1 to 3 R^{#} groups, or
      R^{#} is halogen, cyano, nitro, hydroxy, mercapto, amino, C₁-C₆-alkoxy, C₂-C₆-alkenyloxy, C₂-C₆-alkynyloxy, C₁-C₆-haloalkoxy, C₁-C₆-alkylthio, or C₁-C₆-haloalkylthio, C₁-C₆-alkylsulfonyl, C₁-C₆-alkylsulfinyl, C₁-C₆-alkylamino, di(C₁-C₆alkyl)-amino, C₁-C₆-alkylcarbonyl, C₁-C₆-alkoxycarbonyl, or di(C₁-C₆)-alkylaminocarbonyl;
   formyl, C₁-C₆-alkylcarbonyl, C(═O)NR^{a}R^{b}, CO₂R^{c}, R^{d}, R^{e}, phenyl which may be substituted with 1 to 3 R^{#} groups, or pyridyl which may be substituted with 1 to 3 R^{#} groups,
   R^{a}, R^{b}, R^{c} are each independently hydrogen or C₁-C₄-alkyl which may be substituted with 1 to 3 groups R^{#};
   R^{d} is NRⁱR^{j} or 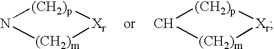
      Rⁱ, R^{j} are each independently hydrogen or C₁-C₄-alkyl which may be substituted with 1 to 3 groups R^{#};
      p, m are each independently 0, 1, 2, or 3, with the proviso that p and m are not both 0.
      X is oxygen, sulfur, amino, C₁-C₄-alkylamino, or phenylamino, or, if p is 0 then X can also be phenoxy or C₁-C₆-alkoxy;
      r is 0 or 1;
   R^{e} is 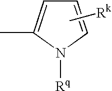
      R^{k}, R^{q} are each independently hydrogen or C₁-C₄-alkyl which may be substituted with 1 to 3 groups R^{#}; or
R¹ and R² may be taken together to form a ring represented by the structure 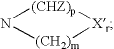
   p, m are 1, 2 or 3;
   X′ is oxygen, sulfur, amino, C₁-C₄-alkylamino, phenylamino, or methylene;
   Z is C₁-C₄-alkyl or phenyl;
R³ is hydrogen, C₁-C₁₀-alkyl, C₂-C₁₁-alkenyl, C₂-C₁₀-alkynyl, C₃-C₁₂-cycloalkyl, wherein the carbon atoms in these groups may be partially or fully halogenated or substituted with
   1 to 3 cyano, nitro, hydroxy, mercapto, amino, C₁-C₆-alkyl, C₃-C₆-cycloalkyl, C₁-C₆-alkoxy, C₁-C₆-alkylamino, di(C₁-C₆-alkyl)-amino, C₁-C₆-alkylthio, C₁-C₆-alkylsulfonyl, or C₁-C₆-alkylsulfinyl groups, wherein the carbon atoms in these groups may be substituted by
      1 to 3 halogen atoms, a 5- to 6-membered aromatic ring system which may contain 1 to 4 heteroatoms selected from oxygen, sulfur and nitrogen and which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms, or
      phenoxy, which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms, or
      a 3- to 6-membered saturated or partially unsaturated ring system which contains 1 to 3 heteroatoms selected from oxygen, sulfur and nitrogen and which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms,
   a 3- to 6-membered saturated or partially unsaturated ring system which contains 1 to 3 heteroatoms selected from oxygen, sulfur and nitrogen and which is unsubstituted or substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, C₁-C₆-haloalkoxy, nitro, or cyano groups, wherein the carbon atoms in these groups may be substituted by 1 to 3 halogen atoms;
R, R⁴ are each independently hydrogen or C₁-C₆-alkyl, C₁-C₆-alkoxycarbonyl, C₁-C₆-alkylaminocarbonyl, or di(C₁-C₆-alkyl)-aminocarbonyl, wherein the carbon atoms in the these groups may be substituted with 1 to 3 groups R^{#};
A is C—R⁵ or N;
B is C—R⁶ or N;
W is C—R⁷ or N;
with the proviso that one of A, B and W is other than N;
   R⁵, R⁶, R⁷ are each independently hydrogen, halogen, nitro, cyano, amino, mercapto, hydroxy, C₁-C₁₀-alkyl, C₂-C₁₀-alkenyl, C₂-C₁₀-alkynyl, C₃-C₆-cycloalkyl, C₁-C₆-alkoxy, C₁-C₆-alkylamino, di(C₁-C₆-alkyl)-amino, C₁-C₆-alkylthio, C₁-C₆-alkylsulfonyl, or C₁-C₆-alkylsulfinyl, wherein the carbon atoms in these groups may be substituted with 1 to 3 groups R^{#}
      a 5- to 6-membered aromatic ringsystem which may contain 1 to 4 heteroatoms selected from oxygen, sulfur and nitrogen and which may be substituted with any combination of 1 to 5 halogen atoms, 1 to 3 C₁-C₆-alkyl, C₁-C₆-haloalkyl, C₁-C₆-alkylthio, C₁-C₆-haloalkylthio, C₁-C₆-alkylsulfonyl, C₁-C₆-alkylsulfinyl, C₁-C₆-haloalkylsulfonyl, C₁-C₆-haloalkylsulfinyl, C₁-C₆-alkoxy, C₁-C₆-haloalkoxy, mercapto, hydroxy, amino, nitro, or cyano groups, wherein the carbon atoms in these groups may be substituted with 1 to 3 groups R^{#};
Y is hydrogen, halogen, cyano, nitro, amino, hydroxy, mercapto, C₁-C₆-alkyl, C₂-C₁₀-alkenyl, C₂-C₁₀-alkynyl, C₃-C₆-cycloalkyl, C₁-C₆-alkoxy, C₁-C₆-alkylamino, di(C₁-C₆)-alkylamino, C₁-C₆-alkylthio, C₁-C₆-alkylsulfonyl, or C₁-C₆-alkylsulfinyl, wherein the carbon atoms in these groups may be substituted with 1 to 3 groups R^{#};
n is 0, 1, or 2;
   or the enantiomers or diastereomers, veterinarily acceptable salts or esters thereof,
   for combating parasites in and on animals.

It is generally a goal of agronomists and veterinarians to possess sufficient means to control parasites, when they attempt to invade or attack animals.

It is an object of the present invention to provide new methods to control parasites in and on animals. Another object of the invention is to provide safer pesticides for animals. Another object of the invention is to provide pesticides for animals that may be used in lower doses than existing pesticides. Another object of the invention is to provide pesticides for animals which provide a long residual control of the parasites.

These objects are met in whole or in part by the present invention.

The invention also relates to compositions containing a parasitically effective amount of compounds of formula I and an acceptable carrier, for combating parasites in and on animals.

The present invention also provides a method for treating, controlling, preventing and protecting animals against infestation and infection by parasites, which comprises orally, topically or parenterally administering or applying to the animals a parasitically effective amount of a compound of formula I or a composition comprising it.

The invention also provides a process for the preparation of a composition for treating, controlling, preventing or protecting animals against infestation or infection by parasites which comprises a parasitically effective amount of a compound of formula I or a composition comprising it.

The insecticidal and acaricidal activity in crop protection of some of the compounds of formula I has been described in EP-A 604 798, and also in J. A Furch et al., “Amidrazones: A New Class of Coleopteran Insecticides”, ACS Symposium Series 686, Am. Chem. Soc., 1998, Chapter 18, p. 178 ff, and also in D. G. Kuhn et al., “Cycloalkyl-substituted Amidrazones: A Novel Class of Insect Control Agents”, ACS Symposium Series 686, Am. Chem. Soc., 1998, Chapter 19, p. 185 ff.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

The compounds of formula I can be prepared according to preparation methods described or referenced in EP-A 604 798 or modifications thereof.

In the definition of formula I shown above, the substituents have the following meanings:

“Halogen” will be taken to mean fluoro, chloro, bromo and iodo.

The term “alkyl” as used herein refers to a branched or unbranched saturated hydrocarbon group having 1 to 10 carbon atoms, especially C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl groups having 1 to 10 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

“Alkoxy” refers to straight-chain or branched alkyl group having 1 to 4 or 6 carbon atoms (as mentioned above) bonded through an oxygen linkage, at any bond in the alkyl group. Examples include methoxy, ethoxy, propoxy, and isopropoxy.

Likewise, the terms “alkylthio”, “alkylamino”, “dialkylamino”, “alkylsulfonyl”, and alkylsulfinyl” refer to straight-chain or branched alkyl group having 1 to 4 or 6 carbon atoms (as mentioned above) bonded through a sulfur-, —NH—, —N—, —S(═O)₂—, or S(═O)— linkage, respectively.

The term “alkenyl” as used herein intends a branched or unbranched unsaturated hydrocarbon group having 3 to 10 carbon atoms and a double bond in any position, such as C₃-C₆ alkenyl such as 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

“Cycloalkyl” refers to a monocyclic 3- to 6-, 8-, 10- or 12-membered saturated carbon atom rings, e.g. C₃-C₈-cycloalkyl such as cyclopropyl, cyclobutyl, cyclopentyl, cyclohexyl, cycloheptyl and cyclooctyl.

A 5- to 6-membered aromatic ring system containing 1 to 4 heteroatoms selected from oxygen, sulfur and nitrogen, intends e.g. 5-membered hetaryl, containing 1 to 4 nitrogen atoms or 1 to 3 nitrogen atoms and 1 sulfur or oxygen atom, e.g. furyl, thienyl, pyrrolyl, isoxazolyl, isothiazolyl, pyrazolyl, oxazolyl, thiazolyl, imidazolyl, oxadiazolyl, thiadiazolyl, oxadiazolyl, triazolyl, and tetrazolyl; or 6-membered hetaryl, containing 1 to 4 nitrogen atoms or 1 to 3 nitrogen atoms and 1 sulfur or oxygen atom, e.g. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

a 3- to 6-membered saturated or partially unsaturated ring system which contains 1 to 3 heteroatoms selected from oxygen, sulfur and nitrogen intends e.g. a saturated 3- to 6-membered ringsystem containing 1 to 3 heteroatoms selected from nitrogen and oxygen, such as aziridine, pyrrolidine, tetrahydrofuran, tetrahydropyran, or piperidine.

By the term “veterinarily acceptable salts” is meant salts the anions of which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

With respect to the intended use of the compounds of formula I, particular preference is given to the following meanings of the substituents, in each case on their own or in combination:

Preference is given to compounds of formula I wherein A denotes C—R⁵.

Moreover, preference is given to compounds of formula I wherein B denotes C—R⁶.

Preference is also given to compounds of formula I wherein W denotes C—R⁷.

Particular preference is given to compounds wherein A denotes C—R⁵, B denotes C—R⁶, and W denotes C—R⁷.

Moreover, preference is given to compounds of formula I wherein R⁵ is halogen or C₁-C₆-haloalkyl, with halogen, especially chlorine, being most preferred.

Preference is also given to compounds of formula I wherein R⁶ is hydrogen or halogen, especially hydrogen.

Preference is further given to compounds of formula I wherein R⁷ is halogen or C₁-C₆-haloalkyl, preferably C₁-C₆-haloalkyl, especially trifluoromethyl.

Moreover, preference is given to compounds of formula I wherein Y is in the ortho-position and is halogen or C₁-C₆-haloalkyl. Particular preference is given to compounds of formula I wherein Y is halogen, especially chlorine.

Preference is given to compounds of formula I wherein n is 1.

Preference is also given to compounds of formula I wherein Q denotes —N═[C(NR¹R²)R³].

Moreover, preference is given to compounds of formula I wherein X¹ is chlorine.

Preference is also given to compounds of formula I wherein R denotes C₁-C₆-alkyl or hydrogen, preferably hydrogen.

Preference is also given to compounds of formula I wherein R¹ and R² each independently are hydrogen, C₁-C₁₀-alkyl which may be substituted by C₁-C₄-alkoxy, or C₃-C₁₀-cycloalkyl which may be substituted with from 1 to 3 halogen.

Moreover, preference is given to compounds of formula I wherein R¹ and R² each independently are hydrogen, C₁-C₄-alkyl, or C₃-C₆-cycloalkyl.

Especially preferred are compounds of formula I wherein R¹ is hydrogen and R² is C₁-C₁₀-alkyl which may be substituted by C₁-C₄-alkoxy, or C₃-C₁₀-cycloalkyl which may be substituted with from 1 to 3 halogen. Compounds of formula I wherein R¹ is hydrogen and R² is C₁-C₄-alkyl or C₃-C₆-cycloalkyl are given special preference.

Moreover, preference is given to compounds of formula I wherein R³ is unsubstituted C₁-C₁₀-alkyl or C₃-C₁₀-cycloalkyl, which may be substituted with 1 to 5 halogen atoms and/or 1 to 3 C₁-C₆-alkyl groups.

Particularly preferred are compounds of formula I wherein R³ is tert.-butyl.

Moreover, particularly preferred are compounds of formula I wherein R³ is cyclopropyl which may be substituted with C₁-C₆-alkyl or halogen, especially 1-methyl-2,2-dichlorocyclopropyl.

Moreover, preference is given to compounds of formula I wherein R⁴ is hydrogen or C₁-C₆-alkyl.

With respect to their use, particular preference is given to the compounds I-A compiled in the tables below. Moreover, the groups mentioned for a substituent in the tables are on their own, independently of the combination in which they are mentioned, a particularly preferred embodiment of the substituent in question.

With respect to their use, particular preference is also given to the hydrochloric acid, maleic acid, dimaleic acid, fumaric acid, difumaric acid, methane sulfenic acid, methane sulfonic acid, and succinic acid adducts of the compounds of the tables below.

Some of the compounds of formula I are new. These are also subject-matter of this invention.

Table 1

Compounds of the formula I-A wherein R³ is methyl and the combination of R¹, R², R⁵, R⁶, R⁷ and Yₙ corresponds in each case to a row of Table A. 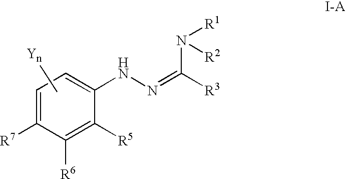
Table 2

Compounds of the formula I-A wherein R³ is ethyl and the combination of R¹, R², R⁵, R⁶, R⁷ and Yₙ corresponds in each case to a row of Table A.

Table 3

Compounds of the formula I-A wherein R³ is ethyl and the combination of R¹, R², R⁵, R⁶, R⁷ and Yₙ corresponds in each case to a row of Table A.

Table 4

Compounds of the formula I-A wherein R³ is propyl and the combination of R¹, R², R⁵, R⁶, R⁷ and Yₙ corresponds in each case to a row of Table A.

Table 5

Compounds of the formula I-A wherein R³ is isopropyl and the combination of R¹, R², R⁵, R⁶, R⁷ and Yₙ corresponds in each case to a row of Table A.

Table 6

Compounds of the formula I-A wherein R³ is isobutyl and the combination of R¹, R², R⁵, R⁶, R⁷ and Yₙ corresponds in each case to a row of Table A.

Table 7

Compounds of the formula I-A wherein R³ is tert-butyl and the combination of R¹, R², R⁵, R⁶, R⁷ and Yₙ corresponds in each case to a row of Table A.

Table 8

Compounds of the formula I-A wherein R³ is neopentyl and the combination of R¹, R², R⁵, R⁶, R⁷ and Yₙ corresponds in each case to a row of Table A.

Table 9

Compounds of the formula I-A wherein R³ is cyclopropyl and the combination of R¹, R², R⁵, R⁶, R⁷ and Yₙ corresponds in each case to a row of Table A.

Table 10

Compounds of the formula I-A wherein R³ is 1,1-dimethyl-propyl and the combination of R¹, R², R⁵, R⁶, R⁷ and Yₙ corresponds in each case to a row of Table A.

Table 11

Compounds of the formula I-A wherein R³ is cyclopropyl and the combination of R¹, R², R⁵, R⁶, R⁷ and Yₙ corresponds in each case to a row of Table A.

Table 12

Compounds of the formula I-A wherein R³ is 2,2-dichloro-cyclopropyl and the combination of R¹, R², R⁵, R⁶, R⁷ and Yₙ corresponds in each case to a row of Table A.

Table 13

Compounds of the formula I-A wherein R³ is 2,2-dibromo-cyclopropyl and the combination of R¹, R², R⁵, R⁶, R⁷ and Yₙ corresponds in each case to a row of Table A.

Table 14

Compounds of the formula I-A wherein R³ is 1-methyl-cyclopropyl and the combination of R¹, R², R⁵, R⁶, R⁷ and Yₙ corresponds in each case to a row of Table A.

Table 15

Compounds of the formula I-A wherein R³ is 1-methyl-2,2-dichlorocyclopropyl and the combination of R¹, R², R⁵, R⁶, R⁷ and Yₙ corresponds in each case to a row of Table A.

Table 16

Compounds of the formula I-A wherein R³ is 1-methyl-2,2-dibromocyclopropyl and the combination of R¹, R², R⁵, R⁶, R⁷ and Yₙ corresponds in each case to a row of Table A.

TABLE A

| No. | R¹ | R² | R⁵ | R⁶ | R⁷ | Yₙ |
|---|---|---|---|---|---|---|
| A-1 | H | H | Cl | H | H | H |
| A-2 | CH₃ | CH₃ | Cl | H | H | H |
| A-3 | CH₂CH₃ | CH₂CH₃ | Cl | H | H | H |
| A-4 | CH₃ | H | Cl | H | H | H |
| A-5 | CH₂CH₃ | H | Cl | H | H | H |
| A-6 | (CH₂)₂CH₃ | H | Cl | H | H | H |
| A-7 | CH(CH₃)₂ | H | Cl | H | H | H |
| A-8 | CH₂CF₃ | H | Cl | H | H | H |
| A-9 | C(CH₃)₃ | H | Cl | H | H | H |
| A-10 | CH₂CH(CH₃)₂ | H | Cl | H | H | H |
| A-11 | CH(CH₃)CH₂CH₃ | H | Cl | H | H | H |
| A-12 | (CH₂)₂OCH₃ | H | Cl | H | H | H |
| A-13 | CH₂CH(OCH₃)₂ | H | Cl | H | H | H |
| A-14 | CH₂CH(OCH₂CH₃)₂ | H | Cl | H | H | H |
| A-15 | CH₂C₆H₅ | H | Cl | H | H | H |
| A-16 | CH(CH₃)C₆H₅ | H | Cl | H | H | H |
| A-17 | (CH₂)₂OC₆H₅ | H | Cl | H | H | H |
| A-18 | (CH₂)₂C₆H₅ | H | Cl | H | H | H |
| A-19 | (CH₂)₂C₆H₄F | H | Cl | H | H | H |
| A-20 | (CH₂)₂C₆H₄Cl | H | Cl | H | H | H |
| A-21 | (CH₂)₂C₆H₄CN | H | Cl | H | H | H |
| A-22 | (CH₂)₂C₆H₄CH₃ | H | Cl | H | H | H |
| A-23 | (CH₂)₂C₆H₄CF₃ | H | Cl | H | H | H |
| A-24 | (CH₂)₂C₆H₄OCH₃ | H | Cl | H | H | H |
| A-25 | (CH₂)₂C₆H₄OCF₃ | H | Cl | H | H | H |
| A-26 | CH₂CH═CH₂ | H | Cl | H | H | H |
| A-27 | cyclo-C₃H₅ | H | Cl | H | H | H |
| A-28 | (CH₂)₂NHCH₃ | H | Cl | H | H | H |
| A-29 | H | H | F | H | H | H |
| A-30 | CH₃ | CH₃ | F | H | H | H |
| A-31 | CH₂CH₃ | CH₂CH₃ | F | H | H | H |
| A-32 | CH₃ | H | F | H | H | H |
| A-33 | CH₂CH₃ | H | F | H | H | H |
| A-34 | (CH₂)₂CH₃ | H | F | H | H | H |
| A-35 | CH(CH₃)₂ | H | F | H | H | H |
| A-36 | CH₂CF₃ | H | F | H | H | H |
| A-37 | C(CH₃)₃ | H | F | H | H | H |
| A-38 | CH₂CH(CH₃)₂ | H | F | H | H | H |
| A-39 | CH(CH₃)CH₂CH₃ | H | F | H | H | H |
| A-40 | (CH₂)₂OCH₃ | H | F | H | H | H |
| A-41 | CH₂CH(OCH₃)₂ | H | F | H | H | H |
| A-42 | CH₂CH(OCH₂CH₃)₂ | H | F | H | H | H |
| A-43 | CH₂C₆H₅ | H | F | H | H | H |
| A-44 | CH(CH₃)C₆H₅ | H | F | H | H | H |
| A-45 | (CH₂)₂OC₆H₅ | H | F | H | H | H |
| A-46 | (CH₂)₂C₆H₅ | H | F | H | H | H |
| A-47 | (CH₂)₂C₆H₄F | H | F | H | H | H |
| A-48 | (CH₂)₂C₆H₄Cl | H | F | H | H | H |
| A-49 | (CH₂)₂C₆H₄CN | H | F | H | H | H |
| A-50 | (CH₂)₂C₆H₄CH₃ | H | F | H | H | H |
| A-51 | (CH₂)₂C₆H₄CF₃ | H | F | H | H | H |
| A-52 | (CH₂)₂C₆H₄OCH₃ | H | F | H | H | H |
| A-53 | (CH₂)₂C₆H₄OCF₃ | H | F | H | H | H |
| A-54 | CH₂CH═CH₂ | H | F | H | H | H |
| A-55 | cyclo-C₃H₅ | H | F | H | H | H |
| A-56 | (CH₂)₂NHCH₃ | H | F | H | H | H |
| A-57 | H | H | CF₃ | H | H | H |
| A-58 | CH₃ | CH₃ | CF₃ | H | H | H |
| A-59 | CH₂CH₃ | CH₂CH₃ | CF₃ | H | H | H |
| A-60 | CH₃ | H | CF₃ | H | H | H |
| A-61 | CH₂CH₃ | H | CF₃ | H | H | H |
| A-62 | (CH₂)₂CH₃ | H | CF₃ | H | H | H |
| A-63 | CH(CH₃)₂ | H | CF₃ | H | H | H |
| A-64 | CH₂CF₃ | H | CF₃ | H | H | H |
| A-65 | C(CH₃)₃ | H | CF₃ | H | H | H |
| A-66 | CH₂CH(CH₃)₂ | H | CF₃ | H | H | H |
| A-67 | CH(CH₃)CH₂CH₃ | H | CF₃ | H | H | H |
| A-68 | (CH₂)₂OCH₃ | H | CF₃ | H | H | H |
| A-69 | CH₂CH(OCH₃)₂ | H | CF₃ | H | H | H |
| A-70 | CH₂CH(OCH₂CH₃)₂ | H | CF₃ | H | H | H |
| A-71 | CH₂C₆H₅ | H | CF₃ | H | H | H |
| A-72 | CH(CH₃)C₆H₅ | H | CF₃ | H | H | H |
| A-73 | (CH₂)₂OC₆H₅ | H | CF₃ | H | H | H |
| A-74 | (CH₂)₂C₆H₅ | H | CF₃ | H | H | H |
| A-75 | (CH₂)₂C₆H₄F | H | CF₃ | H | H | H |
| A-76 | (CH₂)₂C₆H₄Cl | H | CF₃ | H | H | H |
| A-77 | (CH₂)₂C₆H₄CN | H | CF₃ | H | H | H |
| A-78 | (CH₂)₂C₆H₄CH₃ | H | CF₃ | H | H | H |
| A-79 | (CH₂)₂C₆H₄CF₃ | H | CF₃ | H | H | H |
| A-80 | (CH₂)₂C₆H₄OCH₃ | H | CF₃ | H | H | H |
| A-81 | (CH₂)₂C₆H₄OCF₃ | H | CF₃ | H | H | H |
| A-82 | CH₂CH═CH₂ | H | CF₃ | H | H | H |
| A-83 | cyclo-C₃H₅ | H | CF₃ | H | H | H |
| A-84 | (CH₂)₂NHCH₃ | H | CF₃ | H | H | H |
| A-85 | H | H | H | H | Cl | H |
| A-86 | CH₃ | CH₃ | H | H | Cl | H |
| A-87 | CH₂CH₃ | CH₂CH₃ | H | H | Cl | H |
| A-88 | CH₃ | H | H | H | Cl | H |
| A-89 | CH₂CH₃ | H | H | H | Cl | H |
| A-90 | (CH₂)₂CH₃ | H | H | H | Cl | H |
| A-91 | CH(CH₃)₂ | H | H | H | Cl | H |
| A-92 | CH₂CF₃ | H | H | H | Cl | H |
| A-93 | C(CH₃)₃ | H | H | H | Cl | H |
| A-94 | CH₂CH(CH₃)₂ | H | H | H | Cl | H |
| A-95 | CH(CH₃)CH₂CH₃ | H | H | H | Cl | H |
| A-96 | (CH₂)₂OCH₃ | H | H | H | Cl | H |
| A-97 | CH₂CH(OCH₃)₂ | H | H | H | Cl | H |
| A-98 | CH₂CH(OCH₂CH₃)₂ | H | H | H | Cl | H |
| A-99 | CH₂C₆H₅ | H | H | H | Cl | H |
| A-100 | CH(CH₃)C₆H₅ | H | H | H | Cl | H |
| A-101 | (CH₂)₂OC₆H₅ | H | H | H | Cl | H |
| A-102 | (CH₂)₂C₆H₅ | H | H | H | Cl | H |
| A-103 | (CH₂)₂C₆H₄F | H | H | H | Cl | H |
| A-104 | (CH₂)₂C₆H₄Cl | H | H | H | Cl | H |
| A-105 | (CH₂)₂C₆H₄CN | H | H | H | Cl | H |
| A-106 | (CH₂)₂C₆H₄CH₃ | H | H | H | Cl | H |
| A-107 | (CH₂)₂C₆H₄CF₃ | H | H | H | Cl | H |
| A-108 | (CH₂)₂C₆H₄OCH₃ | H | H | H | Cl | H |
| A-109 | (CH₂)₂C₆H₄OCF₃ | H | H | H | Cl | H |
| A-110 | CH₂CH═CH₂ | H | H | H | Cl | H |
| A-111 | cyclo-C₃H₅ | H | H | H | Cl | H |
| A-112 | (CH₂)₂NHCH₃ | H | H | H | Cl | H |
| A-113 | H | H | H | H | F | H |
| A-114 | CH₃ | CH₃ | H | H | F | H |
| A-115 | CH₂CH₃ | CH₂CH₃ | H | H | F | H |
| A-116 | CH₃ | H | H | H | F | H |
| A-117 | CH₂CH₃ | H | H | H | F | H |
| A-118 | (CH₂)₂CH₃ | H | H | H | F | H |
| A-119 | CH(CH₃)₂ | H | H | H | F | H |
| A-120 | CH₂CF₃ | H | H | H | F | H |
| A-121 | C(CH₃)₃ | H | H | H | F | H |
| A-122 | CH₂CH(CH₃)₂ | H | H | H | F | H |
| A-123 | CH(CH₃)CH₂CH₃ | H | H | H | F | H |
| A-124 | (CH₂)₂OCH₃ | H | H | H | F | H |
| A-125 | CH₂CH(OCH₃)₂ | H | H | H | F | H |
| A-126 | CH₂CH(OCH₂CH₃)₂ | H | H | H | F | H |
| A-127 | CH₂C₆H₅ | H | H | H | F | H |
| A-128 | CH(CH₃)C₆H₅ | H | H | H | F | H |
| A-129 | (CH₂)₂OC₆H₅ | H | H | H | F | H |
| A-130 | (CH₂)₂C₆H₅ | H | H | H | F | H |
| A-131 | (CH₂)₂C₆H₄F | H | H | H | F | H |
| A-132 | (CH₂)₂C₆H₄Cl | H | H | H | F | H |
| A-133 | (CH₂)₂C₆H₄CN | H | H | H | F | H |
| A-134 | (CH₂)₂C₆H₄CH₃ | H | H | H | F | H |
| A-135 | (CH₂)₂C₆H₄CF₃ | H | H | H | F | H |
| A-136 | (CH₂)₂C₆H₄OCH₃ | H | H | H | F | H |
| A-137 | (CH₂)₂C₆H₄OCF₃ | H | H | H | F | H |
| A-138 | CH₂CH═CH₂ | H | H | H | F | H |
| A-139 | cyclo-C₃H₅ | H | H | H | F | H |
| A-140 | (CH₂)₂NHCH₃ | H | H | H | F | H |
| A-141 | H | H | H | H | CF₃ | H |
| A-142 | CH₃ | CH₃ | H | H | CF₃ | H |
| A-143 | CH₂CH₃ | CH₂CH₃ | H | H | CF₃ | H |
| A-144 | CH₃ | H | H | H | CF₃ | H |
| A-145 | CH₂CH₃ | H | H | H | CF₃ | H |
| A-146 | (CH₂)₂CH₃ | H | H | H | CF₃ | H |
| A-147 | CH(CH₃)₂ | H | H | H | CF₃ | H |
| A-148 | CH₂CF₃ | H | H | H | CF₃ | H |
| A-149 | C(CH₃)₃ | H | H | H | CF₃ | H |
| A-150 | CH₂CH(CH₃)₂ | H | H | H | CF₃ | H |
| A-151 | CH(CH₃)CH₂CH₃ | H | H | H | CF₃ | H |
| A-152 | (CH₂)₂OCH₃ | H | H | H | CF₃ | H |
| A-153 | CH₂CH(OCH₃)₂ | H | H | H | CF₃ | H |
| A-154 | CH₂CH(OCH₂CH₃)₂ | H | H | H | CF₃ | H |
| A-155 | CH₂C₆H₅ | H | H | H | CF₃ | H |
| A-156 | CH(CH₃)C₆H₅ | H | H | H | CF₃ | H |
| A-157 | (CH₂)₂OC₆H₅ | H | H | H | CF₃ | H |
| A-158 | (CH₂)₂C₆H₅ | H | H | H | CF₃ | H |
| A-159 | (CH₂)₂C₆H₄F | H | H | H | CF₃ | H |
| A-160 | (CH₂)₂C₆H₄Cl | H | H | H | CF₃ | H |
| A-161 | (CH₂)₂C₆H₄CN | H | H | H | CF₃ | H |
| A-162 | (CH₂)₂C₆H₄CH₃ | H | H | H | CF₃ | H |
| A-163 | (CH₂)₂C₆H₄CF₃ | H | H | H | CF₃ | H |
| A-164 | (CH₂)₂C₆H₄OCH₃ | H | H | H | CF₃ | H |
| A-165 | (CH₂)₂C₆H₄OCF₃ | H | H | H | CF₃ | H |
| A-166 | CH₂CH═CH₂ | H | H | H | CF₃ | H |
| A-167 | cyclo-C₃H₅ | H | H | H | CF₃ | H |
| A-168 | (CH₂)₂NHCH₃ | H | H | H | CF₃ | H |
| A-169 | H | H | Cl | H | Cl | H |
| A-170 | CH₃ | CH₃ | Cl | H | Cl | H |
| A-171 | CH₂CH₃ | CH₂CH₃ | Cl | H | Cl | H |
| A-172 | CH₃ | H | Cl | H | Cl | H |
| A-173 | CH₂CH₃ | H | Cl | H | Cl | H |
| A-174 | (CH₂)₂CH₃ | H | Cl | H | Cl | H |
| A-175 | CH(CH₃)₂ | H | Cl | H | Cl | H |
| A-176 | CH₂CF₃ | H | Cl | H | Cl | H |
| A-177 | C(CH₃)₃ | H | Cl | H | Cl | H |
| A-178 | CH₂CH(CH₃)₂ | H | Cl | H | Cl | H |
| A-179 | CH(CH₃)CH₂CH₃ | H | Cl | H | Cl | H |
| A-180 | (CH₂)₂OCH₃ | H | Cl | H | Cl | H |
| A-181 | CH₂CH(OCH₃)₂ | H | Cl | H | Cl | H |
| A-182 | CH₂CH(OCH₂CH₃)₂ | H | Cl | H | Cl | H |
| A-183 | CH₂C₆H₅ | H | Cl | H | Cl | H |
| A-184 | CH(CH₃)C₆H₅ | H | Cl | H | Cl | H |
| A-185 | (CH₂)₂OC₆H₅ | H | Cl | H | Cl | H |
| A-186 | (CH₂)₂C₆H₅ | H | Cl | H | Cl | H |
| A-187 | (CH₂)₂C₆H₄F | H | Cl | H | Cl | H |
| A-188 | (CH₂)₂C₆H₄Cl | H | Cl | H | Cl | H |
| A-189 | (CH₂)₂C₆H₄CN | H | Cl | H | Cl | H |
| A-190 | (CH₂)₂C₆H₄CH₃ | H | Cl | H | Cl | H |
| A-191 | (CH₂)₂C₆H₄CF₃ | H | Cl | H | Cl | H |
| A-192 | (CH₂)₂C₆H₄OCH₃ | H | Cl | H | Cl | H |
| A-193 | (CH₂)₂C₆H₄OCF₃ | H | Cl | H | Cl | H |
| A-194 | CH₂CH═CH₂ | H | Cl | H | Cl | H |
| A-195 | cyclo-C₃H₅ | H | Cl | H | Cl | H |
| A-196 | (CH₂)₂NHCH₃ | H | Cl | H | Cl | H |
| A-197 | H | H | F | H | Cl | H |
| A-198 | CH₃ | CH₃ | F | H | Cl | H |
| A-199 | CH₂CH₃ | CH₂CH₃ | F | H | Cl | H |
| A-200 | CH₃ | H | F | H | Cl | H |
| A-201 | CH₂CH₃ | H | F | H | Cl | H |
| A-202 | (CH₂)₂CH₃ | H | F | H | Cl | H |
| A-203 | CH(CH₃)₂ | H | F | H | Cl | H |
| A-204 | CH₂CF₃ | H | F | H | Cl | H |
| A-205 | C(CH₃)₃ | H | F | H | Cl | H |
| A-206 | CH₂CH(CH₃)₂ | H | F | H | Cl | H |
| A-207 | CH(CH₃)CH₂CH₃ | H | F | H | Cl | H |
| A-208 | (CH₂)₂OCH₃ | H | F | H | Cl | H |
| A-209 | CH₂CH(OCH₃)₂ | H | F | H | Cl | H |
| A-210 | CH₂CH(OCH₂CH₃)₂ | H | F | H | Cl | H |
| A-211 | CH₂C₆H₅ | H | F | H | Cl | H |
| A-212 | CH(CH₃)C₆H₅ | H | F | H | Cl | H |
| A-213 | (CH₂)₂OC₆H₅ | H | F | H | Cl | H |
| A-214 | (CH₂)₂C₆H₅ | H | F | H | Cl | H |
| A-215 | (CH₂)₂C₆H₄F | H | F | H | Cl | H |
| A-216 | (CH₂)₂C₆H₄Cl | H | F | H | Cl | H |
| A-217 | (CH₂)₂C₆H₄CN | H | F | H | Cl | H |
| A-218 | (CH₂)₂C₆H₄CH₃ | H | F | H | Cl | H |
| A-219 | (CH₂)₂C₆H₄CF₃ | H | F | H | Cl | H |
| A-220 | (CH₂)₂C₆H₄OCH₃ | H | F | H | Cl | H |
| A-221 | (CH₂)₂C₆H₄OCF₃ | H | F | H | Cl | H |
| A-222 | CH₂CH═CH₂ | H | F | H | Cl | H |
| A-223 | cyclo-C₃H₅ | H | F | H | Cl | H |
| A-224 | (CH₂)₂NHCH₃ | H | F | H | Cl | H |
| A-225 | H | H | Cl | H | F | H |
| A-226 | CH₃ | CH₃ | Cl | H | F | H |
| A-227 | CH₂CH₃ | CH₂CH₃ | Cl | H | F | H |
| A-228 | CH₃ | H | Cl | H | F | H |
| A-229 | CH₂CH₃ | H | Cl | H | F | H |
| A-230 | (CH₂)₂CH₃ | H | Cl | H | F | H |
| A-231 | CH(CH₃)₂ | H | Cl | H | F | H |
| A-232 | CH₂CF₃ | H | Cl | H | F | H |
| A-233 | C(CH₃)₃ | H | Cl | H | F | H |
| A-234 | CH₂CH(CH₃)₂ | H | Cl | H | F | H |
| A-235 | CH(CH₃)CH₂CH₃ | H | Cl | H | F | H |
| A-236 | (CH₂)₂OCH₃ | H | Cl | H | F | H |
| A-237 | CH₂CH(OCH₃)₂ | H | Cl | H | F | H |
| A-238 | CH₂CH(OCH₂CH₃)₂ | H | Cl | H | F | H |
| A-239 | CH₂C₆H₅ | H | Cl | H | F | H |
| A-240 | CH(CH₃)C₆H₅ | H | Cl | H | F | H |
| A-241 | (CH₂)₂OC₆H₅ | H | Cl | H | F | H |
| A-242 | (CH₂)₂C₆H₅ | H | Cl | H | F | H |
| A-243 | (CH₂)₂C₆H₄F | H | Cl | H | F | H |
| A-244 | (CH₂)₂C₆H₄Cl | H | Cl | H | F | H |
| A-245 | (CH₂)₂C₆H₄CN | H | Cl | H | F | H |
| A-246 | (CH₂)₂C₆H₄CH₃ | H | Cl | H | F | H |
| A-247 | (CH₂)₂C₆H₄CF₃ | H | Cl | H | F | H |
| A-248 | (CH₂)₂C₆H₄OCH₃ | H | Cl | H | F | H |
| A-249 | (CH₂)₂C₆H₄OCF₃ | H | Cl | H | F | H |
| A-250 | CH₂CH═CH₂ | H | Cl | H | F | H |
| A-251 | cyclo-C₃H₅ | H | Cl | H | F | H |
| A-252 | (CH₂)₂NHCH₃ | H | Cl | H | F | H |
| A-253 | H | H | F | H | F | H |
| A-254 | CH₃ | CH₃ | F | H | F | H |
| A-255 | CH₂CH₃ | CH₂CH₃ | F | H | F | H |
| A-256 | CH₃ | H | F | H | F | H |
| A-257 | CH₂CH₃ | H | F | H | F | H |
| A-258 | (CH₂)₂CH₃ | H | F | H | F | H |
| A-259 | CH(CH₃)₂ | H | F | H | F | H |
| A-260 | CH₂CF₃ | H | F | H | F | H |
| A-261 | C(CH₃)₃ | H | F | H | F | H |
| A-262 | CH₂CH(CH₃)₂ | H | F | H | F | H |
| A-263 | CH(CH₃)CH₂CH₃ | H | F | H | F | H |
| A-264 | (CH₂)₂OCH₃ | H | F | H | F | H |
| A-265 | CH₂CH(OCH₃)₂ | H | F | H | F | H |
| A-266 | CH₂CH(OCH₂CH₃)₂ | H | F | H | F | H |
| A-267 | CH₂C₆H₅ | H | F | H | F | H |
| A-268 | CH(CH₃)C₆H₅ | H | F | H | F | H |
| A-269 | (CH₂)₂OC₆H₅ | H | F | H | F | H |
| A-270 | (CH₂)₂C₆H₅ | H | F | H | F | H |
| A-271 | (CH₂)₂C₆H₄F | H | F | H | F | H |
| A-272 | (CH₂)₂C₆H₄Cl | H | F | H | F | H |
| A-273 | (CH₂)₂C₆H₄CN | H | F | H | F | H |
| A-274 | (CH₂)₂C₆H₄CH₃ | H | F | H | F | H |
| A-275 | (CH₂)₂C₆H₄CF₃ | H | F | H | F | H |
| A-276 | (CH₂)₂C₆H₄OCH₃ | H | F | H | F | H |
| A-277 | (CH₂)₂C₆H₄OCF₃ | H | F | H | F | H |
| A-278 | CH₂CH═CH₂ | H | F | H | F | H |
| A-279 | cyclo-C₃H₅ | H | F | H | F | H |
| A-280 | (CH₂)₂NHCH₃ | H | F | H | F | H |
| A-281 | H | H | Cl | H | CF₃ | H |
| A-282 | CH₃ | CH₃ | Cl | H | CF₃ | H |
| A-283 | CH₂CH₃ | CH₂CH₃ | Cl | H | CF₃ | H |
| A-284 | CH₃ | H | Cl | H | CF₃ | H |
| A-285 | CH₂CH₃ | H | Cl | H | CF₃ | H |
| A-286 | (CH₂)₂CH₃ | H | Cl | H | CF₃ | H |
| A-287 | CH(CH₃)₂ | H | Cl | H | CF₃ | H |
| A-288 | CH₂CF₃ | H | Cl | H | CF₃ | H |
| A-289 | C(CH₃)₃ | H | Cl | H | CF₃ | H |
| A-290 | CH₂CH(CH₃)₂ | H | Cl | H | CF₃ | H |
| A-291 | CH(CH₃)CH₂CH₃ | H | Cl | H | CF₃ | H |
| A-292 | (CH₂)₂OCH₃ | H | Cl | H | CF₃ | H |
| A-293 | CH₂CH(OCH₃)₂ | H | Cl | H | CF₃ | H |
| A-294 | CH₂CH(OCH₂CH₃)₂ | H | Cl | H | CF₃ | H |
| A-295 | CH₂C₆H₅ | H | Cl | H | CF₃ | H |
| A-296 | CH(CH₃)C₆H₅ | H | Cl | H | CF₃ | H |
| A-297 | (CH₂)₂OC₆H₅ | H | Cl | H | CF₃ | H |
| A-298 | (CH₂)₂C₆H₅ | H | Cl | H | CF₃ | H |
| A-299 | (CH₂)₂C₆H₄F | H | Cl | H | CF₃ | H |
| A-300 | (CH₂)₂C₆H₄Cl | H | Cl | H | CF₃ | H |
| A-301 | (CH₂)₂C₆H₄CN | H | Cl | H | CF₃ | H |
| A-302 | (CH₂)₂C₆H₄CH₃ | H | Cl | H | CF₃ | H |
| A-303 | (CH₂)₂C₆H₄CF₃ | H | Cl | H | CF₃ | H |
| A-304 | (CH₂)₂C₆H₄OCH₃ | H | Cl | H | CF₃ | H |
| A-305 | (CH₂)₂C₆H₄OCF₃ | H | Cl | H | CF₃ | H |
| A-306 | CH₂CH═CH₂ | H | Cl | H | CF₃ | H |
| A-307 | cyclo-C₃H₅ | H | Cl | H | CF₃ | H |
| A-308 | (CH₂)₂NHCH₃ | H | Cl | H | CF₃ | H |
| A-309 | H | H | CF₃ | H | Cl | H |
| A-310 | CH₃ | CH₃ | CF₃ | H | Cl | H |
| A-311 | CH₂CH₃ | CH₂CH₃ | CF₃ | H | Cl | H |
| A-312 | CH₃ | H | CF₃ | H | Cl | H |
| A-313 | CH₂CH₃ | H | CF₃ | H | Cl | H |
| A-314 | (CH₂)₂CH₃ | H | CF₃ | H | Cl | H |
| A-315 | CH(CH₃)₂ | H | CF₃ | H | Cl | H |
| A-316 | CH₂CF₃ | H | CF₃ | H | Cl | H |
| A-317 | C(CH₃)₃ | H | CF₃ | H | Cl | H |
| A-318 | CH₂CH(CH₃)₂ | H | CF₃ | H | Cl | H |
| A-319 | CH(CH₃)CH₂CH₃ | H | CF₃ | H | Cl | H |
| A-320 | (CH₂)₂OCH₃ | H | CF₃ | H | Cl | H |
| A-321 | CH₂CH(OCH₃)₂ | H | CF₃ | H | Cl | H |
| A-322 | CH₂CH(OCH₂CH₃)₂ | H | CF₃ | H | Cl | H |
| A-323 | CH₂C₆H₅ | H | CF₃ | H | Cl | H |
| A-324 | CH(CH₃)C₆H₅ | H | CF₃ | H | Cl | H |
| A-325 | (CH₂)₂OC₆H₅ | H | CF₃ | H | Cl | H |
| A-326 | (CH₂)₂C₆H₅ | H | CF₃ | H | Cl | H |
| A-327 | (CH₂)₂C₆H₄F | H | CF₃ | H | Cl | H |
| A-328 | (CH₂)₂C₆H₄Cl | H | CF₃ | H | Cl | H |
| A-329 | (CH₂)₂C₆H₄CN | H | CF₃ | H | Cl | H |
| A-330 | (CH₂)₂C₆H₄CH₃ | H | CF₃ | H | Cl | H |
| A-331 | (CH₂)₂C₆H₄CF₃ | H | CF₃ | H | Cl | H |
| A-332 | (CH₂)₂C₆H₄OCH₃ | H | CF₃ | H | Cl | H |
| A-333 | (CH₂)₂C₆H₄OCF₃ | H | CF₃ | H | Cl | H |
| A-334 | CH₂CH═CH₂ | H | CF₃ | H | Cl | H |
| A-335 | cyclo-C₃H₅ | H | CF₃ | H | Cl | H |
| A-336 | (CH₂)₂NHCH₃ | H | CF₃ | H | Cl | H |
| A-337 | H | H | F | H | CF₃ | H |
| A-338 | CH₃ | CH₃ | F | H | CF₃ | H |
| A-339 | CH₂CH₃ | CH₂CH₃ | F | H | CF₃ | H |
| A-340 | CH₃ | H | F | H | CF₃ | H |
| A-341 | CH₂CH₃ | H | F | H | CF₃ | H |
| A-342 | (CH₂)₂CH₃ | H | F | H | CF₃ | H |
| A-343 | CH(CH₃)₂ | H | F | H | CF₃ | H |
| A-344 | CH₂CF₃ | H | F | H | CF₃ | H |
| A-345 | C(CH₃)₃ | H | F | H | CF₃ | H |
| A-346 | CH₂CH(CH₃)₂ | H | F | H | CF₃ | H |
| A-347 | CH(CH₃)CH₂CH₃ | H | F | H | CF₃ | H |
| A-348 | (CH₂)₂OCH₃ | H | F | H | CF₃ | H |
| A-349 | CH₂CH(OCH₃)₂ | H | F | H | CF₃ | H |
| A-350 | CH₂CH(OCH₂CH₃)₂ | H | F | H | CF₃ | H |
| A-351 | CH₂C₆H₅ | H | F | H | CF₃ | H |
| A-352 | CH(CH₃)C₆H₅ | H | F | H | CF₃ | H |
| A-353 | (CH₂)₂OC₆H₅ | H | F | H | CF₃ | H |
| A-354 | (CH₂)₂C₆H₅ | H | F | H | CF₃ | H |
| A-355 | (CH₂)₂C₆H₄F | H | F | H | CF₃ | H |
| A-356 | (CH₂)₂C₆H₄Cl | H | F | H | CF₃ | H |
| A-357 | (CH₂)₂C₆H₄CN | H | F | H | CF₃ | H |
| A-358 | (CH₂)₂C₆H₄CH₃ | H | F | H | CF₃ | H |
| A-359 | (CH₂)₂C₆H₄CF₃ | H | F | H | CF₃ | H |
| A-360 | (CH₂)₂C₆H₄OCH₃ | H | F | H | CF₃ | H |
| A-361 | (CH₂)₂C₆H₄OCF₃ | H | F | H | CF₃ | H |
| A-362 | CH₂CH═CH₂ | H | F | H | CF₃ | H |
| A-363 | cyclo-C₃H₅ | H | F | H | CF₃ | H |
| A-364 | (CH₂)₂NHCH₃ | H | F | H | CF₃ | H |
| A-365 | H | H | CF₃ | H | F | H |
| A-366 | CH₃ | CH₃ | CF₃ | H | F | H |
| A-367 | CH₂CH₃ | CH₂CH₃ | CF₃ | H | F | H |
| A-368 | CH₃ | H | CF₃ | H | F | H |
| A-369 | CH₂CH₃ | H | CF₃ | H | F | H |
| A-370 | (CH₂)₂CH₃ | H | CF₃ | H | F | H |
| A-371 | CH(CH₃)₂ | H | CF₃ | H | F | H |
| A-372 | CH₂CF₃ | H | CF₃ | H | F | H |
| A-373 | C(CH₃)₃ | H | CF₃ | H | F | H |
| A-374 | CH₂CH(CH₃)₂ | H | CF₃ | H | F | H |
| A-375 | CH(CH₃)CH₂CH₃ | H | CF₃ | H | F | H |
| A-376 | (CH₂)₂OCH₃ | H | CF₃ | H | F | H |
| A-377 | CH₂CH(OCH₃)₂ | H | CF₃ | H | F | H |
| A-378 | CH₂CH(OCH₂CH₃)₂ | H | CF₃ | H | F | H |
| A-379 | CH₂C₆H₅ | H | CF₃ | H | F | H |
| A-380 | CH(CH₃)C₆H₅ | H | CF₃ | H | F | H |
| A-381 | (CH₂)₂OC₆H₅ | H | CF₃ | H | F | H |
| A-382 | (CH₂)₂C₆H₅ | H | CF₃ | H | F | H |
| A-383 | (CH₂)₂C₆H₄F | H | CF₃ | H | F | H |
| A-384 | (CH₂)₂C₆H₄Cl | H | CF₃ | H | F | H |
| A-385 | (CH₂)₂C₆H₄CN | H | CF₃ | H | F | H |
| A-386 | (CH₂)₂C₆H₄CH₃ | H | CF₃ | H | F | H |
| A-387 | (CH₂)₂C₆H₄CF₃ | H | CF₃ | H | F | H |
| A-388 | (CH₂)₂C₆H₄OCH₃ | H | CF₃ | H | F | H |
| A-389 | (CH₂)₂C₆H₄OCF₃ | H | CF₃ | H | F | H |
| A-390 | CH₂CH═CH₂ | H | CF₃ | H | F | H |
| A-391 | cyclo-C₃H₅ | H | CF₃ | H | F | H |
| A-392 | (CH₂)₂NHCH₃ | H | CF₃ | H | F | H |
| A-393 | H | H | CF₃ | H | CF₃ | H |
| A-394 | CH₃ | CH₃ | CF₃ | H | CF₃ | H |
| A-395 | CH₂CH₃ | CH₂CH₃ | CF₃ | H | CF₃ | H |
| A-396 | CH₃ | H | CF₃ | H | CF₃ | H |
| A-397 | CH₂CH₃ | H | CF₃ | H | CF₃ | H |
| A-398 | (CH₂)₂CH₃ | H | CF₃ | H | CF₃ | H |
| A-399 | CH(CH₃)₂ | H | CF₃ | H | CF₃ | H |
| A-400 | CH₂CF₃ | H | CF₃ | H | CF₃ | H |
| A-401 | C(CH₃)₃ | H | CF₃ | H | CF₃ | H |
| A-402 | CH₂CH(CH₃)₂ | H | CF₃ | H | CF₃ | H |
| A-403 | CH(CH₃)CH₂CH₃ | H | CF₃ | H | CF₃ | H |
| A-404 | (CH₂)₂OCH₃ | H | CF₃ | H | CF₃ | H |
| A-405 | CH₂CH(OCH₃)₂ | H | CF₃ | H | CF₃ | H |
| A-406 | CH₂CH(OCH₂CH₃)₂ | H | CF₃ | H | CF₃ | H |
| A-407 | CH₂C₆H₅ | H | CF₃ | H | CF₃ | H |
| A-408 | CH(CH₃)C₆H₅ | H | CF₃ | H | CF₃ | H |
| A-409 | (CH₂)₂OC₆H₅ | H | CF₃ | H | CF₃ | H |
| A-410 | (CH₂)₂C₆H₅ | H | CF₃ | H | CF₃ | H |
| A-411 | (CH₂)₂C₆H₄F | H | CF₃ | H | CF₃ | H |
| A-412 | (CH₂)₂C₆H₄Cl | H | CF₃ | H | CF₃ | H |
| A-413 | (CH₂)₂C₆H₄CN | H | CF₃ | H | CF₃ | H |
| A-414 | (CH₂)₂C₆H₄CH₃ | H | CF₃ | H | CF₃ | H |
| A-415 | (CH₂)₂C₆H₄CF₃ | H | CF₃ | H | CF₃ | H |
| A-416 | (CH₂)₂C₆H₄OCH₃ | H | CF₃ | H | CF₃ | H |
| A-417 | (CH₂)₂C₆H₄OCF₃ | H | CF₃ | H | CF₃ | H |
| A-418 | CH₂CH═CH₂ | H | CF₃ | H | CF₃ | H |
| A-419 | cyclo-C₃H₅ | H | CF₃ | H | CF₃ | H |
| A-420 | (CH₂)₂NHCH₃ | H | CF₃ | H | CF₃ | H |
| A-421 | H | H | Cl | Cl | Cl | H |
| A-422 | CH₃ | CH₃ | Cl | Cl | Cl | H |
| A-423 | CH₂CH₃ | CH₂CH₃ | Cl | Cl | Cl | H |
| A-424 | CH₃ | H | Cl | Cl | Cl | H |
| A-425 | CH₂CH₃ | H | Cl | Cl | Cl | H |
| A-426 | (CH₂)₂CH₃ | H | Cl | Cl | Cl | H |
| A-427 | CH(CH₃)₂ | H | Cl | Cl | Cl | H |
| A-428 | CH₂CF₃ | H | Cl | Cl | Cl | H |
| A-429 | C(CH₃)₃ | H | Cl | Cl | Cl | H |
| A-430 | CH₂CH(CH₃)₂ | H | Cl | Cl | Cl | H |
| A-431 | CH(CH₃)CH₂CH₃ | H | Cl | Cl | Cl | H |
| A-432 | (CH₂)₂OCH₃ | H | Cl | Cl | Cl | H |
| A-433 | CH₂CH(OCH₃)₂ | H | Cl | Cl | Cl | H |
| A-434 | CH₂CH(OCH₂CH₃)₂ | H | Cl | Cl | Cl | H |
| A-435 | CH₂C₆H₅ | H | Cl | Cl | Cl | H |
| A-436 | CH(CH₃)C₆H₅ | H | Cl | Cl | Cl | H |
| A-437 | (CH₂)₂OC₆H₅ | H | Cl | Cl | Cl | H |
| A-438 | (CH₂)₂C₆H₅ | H | Cl | Cl | Cl | H |
| A-439 | (CH₂)₂C₆H₄F | H | Cl | Cl | Cl | H |
| A-440 | (CH₂)₂C₆H₄Cl | H | Cl | Cl | Cl | H |
| A-441 | (CH₂)₂C₆H₄CN | H | Cl | Cl | Cl | H |
| A-442 | (CH₂)₂C₆H₄CH₃ | H | Cl | Cl | Cl | H |
| A-443 | (CH₂)₂C₆H₄CF₃ | H | Cl | Cl | Cl | H |
| A-444 | (CH₂)₂C₆H₄OCH₃ | H | Cl | Cl | Cl | H |
| A-445 | (CH₂)₂C₆H₄OCF₃ | H | Cl | Cl | Cl | H |
| A-446 | CH₂CH═CH₂ | H | Cl | Cl | Cl | H |
| A-447 | cyclo-C₃H₅ | H | Cl | Cl | Cl | H |
| A-448 | (CH₂)₂NHCH₃ | H | Cl | Cl | Cl | H |
| A-449 | H | H | Cl | F | Cl | H |
| A-450 | CH₃ | CH₃ | Cl | F | Cl | H |
| A-451 | CH₂CH₃ | CH₂CH₃ | Cl | F | Cl | H |
| A-452 | CH₃ | H | Cl | F | Cl | H |
| A-453 | CH₂CH₃ | H | Cl | F | Cl | H |
| A-454 | (CH₂)₂CH₃ | H | Cl | F | Cl | H |
| A-455 | CH(CH₃)₂ | H | Cl | F | Cl | H |
| A-456 | CH₂CF₃ | H | Cl | F | Cl | H |
| A-457 | C(CH₃)₃ | H | Cl | F | Cl | H |
| A-458 | CH₂CH(CH₃)₂ | H | Cl | F | Cl | H |
| A-459 | CH(CH₃)CH₂CH₃ | H | Cl | F | Cl | H |
| A-460 | (CH₂)₂OCH₃ | H | Cl | F | Cl | H |
| A-461 | CH₂CH(OCH₃)₂ | H | Cl | F | Cl | H |
| A-462 | CH₂CH(OCH₂CH₃)₂ | H | Cl | F | Cl | H |
| A-463 | CH₂C₆H₅ | H | Cl | F | Cl | H |
| A-464 | CH(CH₃)C₆H₅ | H | Cl | F | Cl | H |
| A-465 | (CH₂)₂OC₆H₅ | H | Cl | F | Cl | H |
| A-466 | (CH₂)₂C₆H₅ | H | Cl | F | Cl | H |
| A-467 | (CH₂)₂C₆H₄F | H | Cl | F | Cl | H |
| A-468 | (CH₂)₂C₆H₄Cl | H | Cl | F | Cl | H |
| A-469 | (CH₂)₂C₆H₄CN | H | Cl | F | Cl | H |
| A-470 | (CH₂)₂C₆H₄CH₃ | H | Cl | F | Cl | H |
| A-471 | (CH₂)₂C₆H₄CF₃ | H | Cl | F | Cl | H |
| A-472 | (CH₂)₂C₆H₄OCH₃ | H | Cl | F | Cl | H |
| A-473 | (CH₂)₂C₆H₄OCF₃ | H | Cl | F | Cl | H |
| A-474 | CH₂CH═CH₂ | H | Cl | F | Cl | H |
| A-475 | cyclo-C₃H₅ | H | Cl | F | Cl | H |
| A-476 | (CH₂)₂NHCH₃ | H | Cl | F | Cl | H |
| A-477 | H | H | Cl | Cl | F | H |
| A-478 | CH₃ | CH₃ | Cl | Cl | F | H |
| A-479 | CH₂CH₃ | CH₂CH₃ | Cl | Cl | F | H |
| A-480 | CH₃ | H | Cl | Cl | F | H |
| A-481 | CH₂CH₃ | H | Cl | Cl | F | H |
| A-482 | (CH₂)₂CH₃ | H | Cl | Cl | F | H |
| A-483 | CH(CH₃)₂ | H | Cl | Cl | F | H |
| A-484 | CH₂CF₃ | H | Cl | Cl | F | H |
| A-485 | C(CH₃)₃ | H | Cl | Cl | F | H |
| A-486 | CH₂CH(CH₃)₂ | H | Cl | Cl | F | H |
| A-487 | CH(CH₃)CH₂CH₃ | H | Cl | Cl | F | H |
| A-488 | (CH₂)₂OCH₃ | H | Cl | Cl | F | H |
| A-489 | CH₂CH(OCH₃)₂ | H | Cl | Cl | F | H |
| A-490 | CH₂CH(OCH₂CH₃)₂ | H | Cl | Cl | F | H |
| A-491 | CH₂C₆H₅ | H | Cl | Cl | F | H |
| A-492 | CH(CH₃)C₆H₅ | H | Cl | Cl | F | H |
| A-493 | (CH₂)₂OC₆H₅ | H | Cl | Cl | F | H |
| A-494 | (CH₂)₂C₆H₅ | H | Cl | Cl | F | H |
| A-495 | (CH₂)₂C₆H₄F | H | Cl | Cl | F | H |
| A-496 | (CH₂)₂C₆H₄Cl | H | Cl | Cl | F | H |
| A-497 | (CH₂)₂C₆H₄CN | H | Cl | Cl | F | H |
| A-498 | (CH₂)₂C₆H₄CH₃ | H | Cl | Cl | F | H |
| A-499 | (CH₂)₂C₆H₄CF₃ | H | Cl | Cl | F | H |
| A-500 | (CH₂)₂C₆H₄OCH₃ | H | Cl | Cl | F | H |
| A-501 | (CH₂)₂C₆H₄OCF₃ | H | Cl | Cl | F | H |
| A-502 | CH₂CH═CH₂ | H | Cl | Cl | F | H |
| A-503 | cyclo-C₃H₅ | H | Cl | Cl | F | H |
| A-504 | (CH₂)₂NHCH₃ | H | Cl | Cl | F | H |
| A-505 | H | H | F | Cl | Cl | H |
| A-506 | CH₃ | CH₃ | F | Cl | Cl | H |
| A-507 | CH₂CH₃ | CH₂CH₃ | F | Cl | Cl | H |
| A-508 | CH₃ | H | F | Cl | Cl | H |
| A-509 | CH₂CH₃ | H | F | Cl | Cl | H |
| A-510 | (CH₂)₂CH₃ | H | F | Cl | Cl | H |
| A-511 | CH(CH₃)₂ | H | F | Cl | Cl | H |
| A-512 | CH₂CF₃ | H | F | Cl | Cl | H |
| A-513 | C(CH₃)₃ | H | F | Cl | Cl | H |
| A-514 | CH₂CH(CH₃)₂ | H | F | Cl | Cl | H |
| A-515 | CH(CH₃)CH₂CH₃ | H | F | Cl | Cl | H |
| A-516 | (CH₂)₂OCH₃ | H | F | Cl | Cl | H |
| A-517 | CH₂CH(OCH₃)₂ | H | F | Cl | Cl | H |
| A-518 | CH₂CH(OCH₂CH₃)₂ | H | F | Cl | Cl | H |
| A-519 | CH₂C₆H₅ | H | F | Cl | Cl | H |
| A-520 | CH(CH₃)C₆H₅ | H | F | Cl | Cl | H |
| A-521 | (CH₂)₂OC₆H₅ | H | F | Cl | Cl | H |
| A-522 | (CH₂)₂C₆H₅ | H | F | Cl | Cl | H |
| A-523 | (CH₂)₂C₆H₄F | H | F | Cl | Cl | H |
| A-524 | (CH₂)₂C₆H₄Cl | H | F | Cl | Cl | H |
| A-525 | (CH₂)₂C₆H₄CN | H | F | Cl | Cl | H |
| A-526 | (CH₂)₂C₆H₄CH₃ | H | F | Cl | Cl | H |
| A-527 | (CH₂)₂C₆H₄CF₃ | H | F | Cl | Cl | H |
| A-528 | (CH₂)₂C₆H₄OCH₃ | H | F | Cl | Cl | H |
| A-529 | (CH₂)₂C₆H₄OCF₃ | H | F | Cl | Cl | H |
| A-530 | CH₂CH═CH₂ | H | F | Cl | Cl | H |
| A-531 | cyclo-C₃H₅ | H | F | Cl | Cl | H |
| A-532 | (CH₂)₂NHCH₃ | H | F | Cl | Cl | H |
| A-533 | H | H | F | F | Cl | H |
| A-534 | CH₃ | ClH₃ | F | F | Cl | H |
| A-535 | CH₂CH₃ | CH₂CH₃ | F | F | Cl | H |
| A-536 | CH₃ | H | F | F | Cl | H |
| A-537 | CH₂CH₃ | H | F | F | Cl | H |
| A-538 | (CH₂)₂CH₃ | H | F | F | Cl | H |
| A-539 | CH(CH₃)₂ | H | F | F | Cl | H |
| A-540 | CH₂CF₃ | H | F | F | Cl | H |
| A-541 | C(CH₃)₃ | H | F | F | Cl | H |
| A-542 | CH₂CH(CH₃)₂ | H | F | F | Cl | H |
| A-543 | CH(CH₃)CH₂CH₃ | H | F | F | Cl | H |
| A-544 | (CH₂)₂OCH₃ | H | F | F | Cl | H |
| A-545 | CH₂CH(OCH₃)₂ | H | F | F | Cl | H |
| A-546 | CH₂CH(OCH₂CH₃)₂ | H | F | F | Cl | H |
| A-547 | CH₂C₆H₅ | H | F | F | Cl | H |
| A-548 | CH(CH₃)C₆H₅ | H | F | F | Cl | H |
| A-549 | (CH₂)₂OC₆H₅ | H | F | F | Cl | H |
| A-550 | (CH₂)₂C₆H₅ | H | F | F | Cl | H |
| A-551 | (CH₂)₂C₆H₄F | H | F | F | Cl | H |
| A-552 | (CH₂)₂C₆H₄Cl | H | F | F | Cl | H |
| A-553 | (CH₂)₂C₆H₄CN | H | F | F | Cl | H |
| A-554 | (CH₂)₂C₆H₄CH₃ | H | F | F | Cl | H |
| A-555 | (CH₂)₂C₆H₄CF₃ | H | F | F | Cl | H |
| A-556 | (CH₂)₂C₆H₄OCH₃ | H | F | F | Cl | H |
| A-557 | (CH₂)₂C₆H₄OCF₃ | H | F | F | Cl | H |
| A-558 | CH₂CH═CH₂ | H | F | F | Cl | H |
| A-559 | cyclo-C₃H₅ | H | F | F | Cl | H |
| A-560 | (CH₂)₂NHCH₃ | H | F | F | Cl | H |
| A-561 | H | H | F | Cl | F | H |
| A-562 | CH₃ | CH₃ | F | Cl | F | H |
| A-563 | CH₂CH₃ | CH₂CH₃ | F | Cl | F | H |
| A-564 | CH₃ | H | F | Cl | F | H |
| A-565 | CH₂CH₃ | H | F | Cl | F | H |
| A-566 | (CH₂)₂CH₃ | H | F | Cl | F | H |
| A-567 | CH(CH₃)₂ | H | F | Cl | F | H |
| A-568 | CH₂CF₃ | H | F | Cl | F | H |
| A-569 | C(CH₃)₃ | H | F | Cl | F | H |
| A-570 | CH₂CH(CH₃)₂ | H | F | Cl | F | H |
| A-571 | CH(CH₃)CH₂CH₃ | H | F | Cl | F | H |
| A-572 | (CH₂)₂OCH₃ | H | F | Cl | F | H |
| A-573 | CH₂CH(OCH₃)₂ | H | F | Cl | F | H |
| A-574 | CH₂CH(OCH₂CH₃)₂ | H | F | Cl | F | H |
| A-575 | CH₂C₆H₅ | H | F | Cl | F | H |
| A-576 | CH(CH₃)C₆H₅ | H | F | Cl | F | H |
| A-577 | (CH₂)₂OC₆H₅ | H | F | Cl | F | H |
| A-578 | (CH₂)₂C₆H₅ | H | F | Cl | F | H |
| A-579 | (CH₂)₂C₆H₄F | H | F | Cl | F | H |
| A-580 | (CH₂)₂C₆H₄Cl | H | F | Cl | F | H |
| A-581 | (CH₂)₂C₆H₄CN | H | F | Cl | F | H |
| A-582 | (CH₂)₂C₆H₄CH₃ | H | F | Cl | F | H |
| A-583 | (CH₂)₂C₆H₄CF₃ | H | F | Cl | F | H |
| A-584 | (CH₂)₂C₆H₄OCH₃ | H | F | Cl | F | H |
| A-585 | (CH₂)₂C₆H₄OCF₃ | H | F | Cl | F | H |
| A-586 | CH₂CH═CH₂ | H | F | Cl | F | H |
| A-587 | cyclo-C₃H₅ | H | F | Cl | F | H |
| A-588 | (CH₂)₂NHCH₃ | H | F | Cl | F | H |
| A-589 | H | H | Cl | F | F | H |
| A-590 | CH₃ | CH₃ | Cl | F | F | H |
| A-591 | CH₂CH₃ | CH₂CH₃ | Cl | F | F | H |
| A-592 | CH₃ | H | Cl | F | F | H |
| A-593 | CH₂CH₃ | H | Cl | F | F | H |
| A-594 | (CH₂)₂CH₃ | H | Cl | F | F | H |
| A-595 | CH(CH₃)₂ | H | Cl | F | F | H |
| A-596 | CH₂CF₃ | H | Cl | F | F | H |
| A-597 | C(CH₃)₃ | H | Cl | F | F | H |
| A-598 | CH₂CH(CH₃)₂ | H | Cl | F | F | H |
| A-599 | CH(CH₃)CH₂CH₃ | H | Cl | F | F | H |
| A-600 | (CH₂)₂OCH₃ | H | Cl | F | F | H |
| A-601 | CH₂CH(OCH₃)₂ | H | Cl | F | F | H |
| A-602 | CH₂CH(OCH₂CH₃)₂ | H | Cl | F | F | H |
| A-603 | CH₂C₆H₅ | H | Cl | F | F | H |
| A-604 | CH(CH₃)C₆H₅ | H | Cl | F | F | H |
| A-605 | (CH₂)₂OC₆H₅ | H | Cl | F | F | H |
| A-606 | (CH₂)₂C₆H₅ | H | Cl | F | F | H |
| A-607 | (CH₂)₂C₆H₄F | H | Cl | F | F | H |
| A-608 | (CH₂)₂C₆H₄Cl | H | Cl | F | F | H |
| A-609 | (CH₂)₂C₆H₄CN | H | Cl | F | F | H |
| A-610 | (CH₂)₂C₆H₄CH₃ | H | Cl | F | F | H |
| A-611 | (CH₂)₂C₆H₄CF₃ | H | Cl | F | F | H |
| A-612 | (CH₂)₂C₆H₄OCH₃ | H | Cl | F | F | H |
| A-613 | (CH₂)₂C₆H₄OCF₃ | H | Cl | F | F | H |
| A-614 | CH₂CH═CH₂ | H | Cl | F | F | H |
| A-615 | cyclo-C₃H₅ | H | Cl | F | F | H |
| A-616 | (CH₂)₂NHCH₃ | H | Cl | F | F | H |
| A-617 | H | H | F | F | F | H |
| A-618 | CH₃ | CH₃ | F | F | F | H |
| A-619 | CH₂CH₃ | CH₂CH₃ | F | F | F | H |
| A-620 | CH₃ | H | F | F | F | H |
| A-621 | CH₂CH₃ | H | F | F | F | H |
| A-622 | (CH₂)₂CH₃ | H | F | F | F | H |
| A-623 | CH(CH₃)₂ | H | F | F | F | H |
| A-624 | CH₂CF₃ | H | F | F | F | H |
| A-625 | C(CH₃)₃ | H | F | F | F | H |
| A-626 | CH₂CH(CH₃)₂ | H | F | F | F | H |
| A-627 | CH(CH₃)CH₂CH₃ | H | F | F | F | H |
| A-628 | (CH₂)₂OCH₃ | H | F | F | F | H |
| A-629 | CH₂CH(OCH₃)₂ | H | F | F | F | H |
| A-630 | CH₂CH(OCH₂CH₃)₂ | H | F | F | F | H |
| A-631 | CH₂C₆H₅ | H | F | F | F | H |
| A-632 | CH(CH₃)C₆H₅ | H | F | F | F | H |
| A-633 | (CH₂)₂OC₆H₅ | H | F | F | F | H |
| A-634 | (CH₂)₂C₆H₅ | H | F | F | F | H |
| A-635 | (CH₂)₂C₆H₄F | H | F | F | F | H |
| A-636 | (CH₂)₂C₆H₄Cl | H | F | F | F | H |
| A-637 | (CH₂)₂C₆H₄CN | H | F | F | F | H |
| A-638 | (CH₂)₂C₆H₄CH₃ | H | F | F | F | H |
| A-639 | (CH₂)₂C₆H₄CF₃ | H | F | F | F | H |
| A-640 | (CH₂)₂C₆H₄OCH₃ | H | F | F | F | H |
| A-641 | (CH₂)₂C₆H₄OCF₃ | H | F | F | F | H |
| A-642 | CH₂CH═CH₂ | H | F | F | F | H |
| A-643 | cyclo-C₃H₅ | H | F | F | F | H |
| A-644 | (CH₂)₂NHCH₃ | H | F | F | F | H |
| A-645 | H | H | Cl | Cl | CF₃ | H |
| A-646 | CH₃ | CH₃ | Cl | Cl | CF₃ | H |
| A-647 | CH₂CH₃ | CH₂CH₃ | Cl | Cl | CF₃ | H |
| A-648 | CH₃ | H | Cl | Cl | CF₃ | H |
| A-649 | CH₂CH₃ | H | Cl | Cl | CF₃ | H |
| A-650 | (CH₂)₂CH₃ | H | Cl | Cl | CF₃ | H |
| A-651 | CH(CH₃)₂ | H | Cl | Cl | CF₃ | H |
| A-652 | CH₂CF₃ | H | Cl | Cl | CF₃ | H |
| A-653 | C(CH₃)₃ | H | Cl | Cl | CF₃ | H |
| A-654 | CH₂CH(CH₃)₂ | H | Cl | Cl | CF₃ | H |
| A-655 | CH(CH₃)CH₂CH₃ | H | Cl | Cl | CF₃ | H |
| A-656 | (CH₂)₂OCH₃ | H | Cl | Cl | CF₃ | H |
| A-657 | CH₂CH(OCH₃)₂ | H | Cl | Cl | CF₃ | H |
| A-658 | CH₂CH(OCH₂CH₃)₂ | H | Cl | Cl | CF₃ | H |
| A-659 | CH₂C₆H₅ | H | Cl | Cl | CF₃ | H |
| A-660 | CH(CH₃)C₆H₅ | H | Cl | Cl | CF₃ | H |
| A-661 | (CH₂)₂OC₆H₅ | H | Cl | Cl | CF₃ | H |
| A-662 | (CH₂)₂C₆H₅ | H | Cl | Cl | CF₃ | H |
| A-663 | (CH₂)₂C₆H₄F | H | Cl | Cl | CF₃ | H |
| A-664 | (CH₂)₂C₆H₄Cl | H | Cl | Cl | CF₃ | H |
| A-665 | (CH₂)₂C₆H₄CN | H | Cl | Cl | CF₃ | H |
| A-666 | (CH₂)₂C₆H₄CH₃ | H | Cl | Cl | CF₃ | H |
| A-667 | (CH₂)₂C₆H₄CF₃ | H | Cl | Cl | CF₃ | H |
| A-668 | (CH₂)₂C₆H₄OCH₃ | H | Cl | Cl | CF₃ | H |
| A-669 | (CH₂)₂C₆H₄OCF₃ | H | Cl | Cl | CF₃ | H |
| A-670 | CH₂CH═CH₂ | H | Cl | Cl | CF₃ | H |
| A-671 | cyclo-C₃H₅ | H | Cl | Cl | CF₃ | H |
| A-672 | (CH₂)₂NHCH₃ | H | Cl | Cl | CF₃ | H |
| A-673 | H | H | CF₃ | Cl | Cl | H |
| A-674 | CH₃ | CH₃ | CF₃ | Cl | Cl | H |
| A-675 | CH₂CH₃ | CH₂CH₃ | CF₃ | Cl | Cl | H |
| A-676 | CH₃ | H | CF₃ | Cl | Cl | H |
| A-677 | CH₂CH₃ | H | CF₃ | Cl | Cl | H |
| A-678 | (CH₂)₂CH₃ | H | CF₃ | Cl | Cl | H |
| A-679 | CH(CH₃)₂ | H | CF₃ | Cl | Cl | H |
| A-680 | CH₂CF₃ | H | CF₃ | Cl | Cl | H |
| A-681 | C(CH₃)₃ | H | CF₃ | Cl | Cl | H |
| A-682 | CH₂CH(CH₃)₂ | H | CF₃ | Cl | Cl | H |
| A-683 | CH(CH₃)CH₂CH₃ | H | CF₃ | Cl | Cl | H |
| A-684 | (CH₂)₂OCH₃ | H | CF₃ | Cl | Cl | H |
| A-685 | CH₂CH(OCH₃)₂ | H | CF₃ | Cl | Cl | H |
| A-686 | CH₂CH(OCH₂CH₃)₂ | H | CF₃ | Cl | Cl | H |
| A-687 | CH₂C₆H₅ | H | CF₃ | Cl | Cl | H |
| A-688 | CH(CH₃)C₆H₅ | H | CF₃ | Cl | Cl | H |
| A-689 | (CH₂)₂OC₆H₅ | H | CF₃ | Cl | Cl | H |
| A-690 | (CH₂)₂C₆H₅ | H | CF₃ | Cl | Cl | H |
| A-691 | (CH₂)₂C₆H₄F | H | CF₃ | Cl | Cl | H |
| A-692 | (CH₂)₂C₆H₄Cl | H | CF₃ | Cl | Cl | H |
| A-693 | (CH₂)₂C₆H₄CN | H | CF₃ | Cl | Cl | H |
| A-694 | (CH₂)₂C₆H₄CH₃ | H | CF₃ | Cl | Cl | H |
| A-695 | (CH₂)₂C₆H₄CF₃ | H | CF₃ | Cl | Cl | H |
| A-696 | (CH₂)₂C₆H₄OCH₃ | H | CF₃ | Cl | Cl | H |
| A-697 | (CH₂)₂C₆H₄OCF₃ | H | CF₃ | Cl | Cl | H |
| A-698 | CH₂CH═CH₂ | H | CF₃ | Cl | Cl | H |
| A-699 | cyclo-C₃H₅ | H | CF₃ | Cl | Cl | H |
| A-700 | (CH₂)₂NHCH₃ | H | CF₃ | Cl | Cl | H |
| A-701 | H | H | CF₃ | Cl | F | H |
| A-702 | CH₃ | CH₃ | CF₃ | Cl | F | H |
| A-703 | CH₂CH₃ | CH₂CH₃ | CF₃ | Cl | F | H |
| A-704 | CH₃ | H | CF₃ | Cl | F | H |
| A-705 | CH₂CH₃ | H | CF₃ | Cl | F | H |
| A-706 | (CH₂)₂CH₃ | H | CF₃ | Cl | F | H |
| A-707 | CH(CH₃)₂ | H | CF₃ | Cl | F | H |
| A-708 | CH₂CF₃ | H | CF₃ | Cl | F | H |
| A-709 | C(CH₃)₃ | H | CF₃ | Cl | F | H |
| A-710 | CH₂CH(CH₃)₂ | H | CF₃ | Cl | F | H |
| A-711 | CH(CH₃)CH₂CH₃ | H | CF₃ | Cl | F | H |
| A-712 | (CH₂)₂OCH₃ | H | CF₃ | Cl | F | H |
| A-713 | CH₂CH(OCH₃)₂ | H | CF₃ | Cl | F | H |
| A-714 | CH₂CH(OCH₂CH₃)₂ | H | CF₃ | Cl | F | H |
| A-715 | CH₂C₆H₅ | H | CF₃ | Cl | F | H |
| A-716 | CH(CH₃)C₆H₅ | H | CF₃ | Cl | F | H |
| A-717 | (CH₂)₂OC₆H₅ | H | CF₃ | Cl | F | H |
| A-718 | (CH₂)₂C₆H₅ | H | CF₃ | Cl | F | H |
| A-719 | (CH₂)₂C₆H₄F | H | CF₃ | Cl | F | H |
| A-720 | (CH₂)₂C₆H₄Cl | H | CF₃ | Cl | F | H |
| A-721 | (CH₂)₂C₆H₄CN | H | CF₃ | Cl | F | H |
| A-722 | (CH₂)₂C₆H₄CH₃ | H | CF₃ | Cl | F | H |
| A-723 | (CH₂)₂C₆H₄CF₃ | H | CF₃ | Cl | F | H |
| A-724 | (CH₂)₂C₆H₄OCH₃ | H | CF₃ | Cl | F | H |
| A-725 | (CH₂)₂C₆H₄OCF₃ | H | CF₃ | Cl | F | H |
| A-726 | CH₂CH═CH₂ | H | CF₃ | Cl | F | H |
| A-727 | cyclo-C₃H₅ | H | CF₃ | Cl | F | H |
| A-728 | (CH₂)₂NHCH₃ | H | CF₃ | Cl | F | H |
| A-729 | H | H | F | Cl | CF₃ | H |
| A-730 | CH₃ | CH₃ | F | Cl | CF₃ | H |
| A-731 | CH₂CH₃ | CH₂CH₃ | F | Cl | CF₃ | H |
| A-732 | CH₃ | H | F | Cl | CF₃ | H |
| A-733 | CH₂CH₃ | H | F | Cl | CF₃ | H |
| A-734 | (CH₂)₂CH₃ | H | F | Cl | CF₃ | H |
| A-735 | CH(CH₃)₂ | H | F | Cl | CF₃ | H |
| A-736 | CH₂CF₃ | H | F | Cl | CF₃ | H |
| A-737 | C(CH₃)₃ | H | F | Cl | CF₃ | H |
| A-738 | CH₂CH(CH₃)₂ | H | F | Cl | CF₃ | H |
| A-739 | CH(CH₃)CH₂CH₃ | H | F | Cl | CF₃ | H |
| A-740 | (CH₂)₂OCH₃ | H | F | Cl | CF₃ | H |
| A-741 | CH₂CH(OCH₃)₂ | H | F | Cl | CF₃ | H |
| A-742 | CH₂CH(OCH₂CH₃)₂ | H | F | Cl | CF₃ | H |
| A-743 | CH₂C₆H₅ | H | F | Cl | CF₃ | H |
| A-744 | CH(CH₃)C₆H₅ | H | F | Cl | CF₃ | H |
| A-745 | (CH₂)₂OC₆H₅ | H | F | Cl | CF₃ | H |
| A-746 | (CH₂)₂C₆H₅ | H | F | Cl | CF₃ | H |
| A-747 | (CH₂)₂C₆H₄F | H | F | Cl | CF₃ | H |
| A-748 | (CH₂)₂C₆H₄Cl | H | F | Cl | CF₃ | H |
| A-749 | (CH₂)₂C₆H₄CN | H | F | Cl | CF₃ | H |
| A-750 | (CH₂)₂C₆H₄CH₃ | H | F | Cl | CF₃ | H |
| A-751 | (CH₂)₂C₆H₄CF₃ | H | F | Cl | CF₃ | H |
| A-752 | (CH₂)₂C₆H₄OCH₃ | H | F | Cl | CF₃ | H |
| A-753 | (CH₂)₂C₆H₄OCF₃ | H | F | Cl | CF₃ | H |
| A-754 | CH₂CH═CH₂ | H | F | Cl | CF₃ | H |
| A-755 | cyclo-C₃H₅ | H | F | Cl | CF₃ | H |
| A-756 | (CH₂)₂NHCH₃ | H | F | Cl | CF₃ | H |
| A-757 | H | H | F | Cl | CF₃ | H |
| A-758 | CH₃ | CH₃ | F | Cl | CF₃ | H |
| A-759 | CH₂CH₃ | CH₂CH₃ | F | Cl | CF₃ | H |
| A-760 | CH₃ | H | F | Cl | CF₃ | H |
| A-761 | CH₂CH₃ | H | F | Cl | CF₃ | H |
| A-762 | (CH₂)₂CH₃ | H | F | Cl | CF₃ | H |
| A-763 | CH(CH₃)₂ | H | F | Cl | CF₃ | H |
| A-764 | CH₂CF₃ | H | F | Cl | CF₃ | H |
| A-765 | C(CH₃)₃ | H | F | Cl | CF₃ | H |
| A-766 | CH₂CH(CH₃)₂ | H | F | Cl | CF₃ | H |
| A-767 | CH(CH₃)CH₂CH₃ | H | F | Cl | CF₃ | H |
| A-768 | (CH₂)₂OCH₃ | H | F | Cl | CF₃ | H |
| A-769 | CH₂CH(OCH₃)₂ | H | F | Cl | CF₃ | H |
| A-770 | CH₂CH(OCH₂CH₃)₂ | H | F | Cl | CF₃ | H |
| A-771 | CH₂C₆H₅ | H | F | Cl | CF₃ | H |
| A-772 | CH(CH₃)C₆H₅ | H | F | Cl | CF₃ | H |
| A-773 | (CH₂)₂OC₆H₅ | H | F | Cl | CF₃ | H |
| A-774 | (CH₂)₂C₆H₅ | H | F | Cl | CF₃ | H |
| A-775 | (CH₂)₂C₆H₄F | H | F | Cl | CF₃ | H |
| A-776 | (CH₂)₂C₆H₄Cl | H | F | Cl | CF₃ | H |
| A-777 | (CH₂)₂C₆H₄CN | H | F | Cl | CF₃ | H |
| A-778 | (CH₂)₂C₆H₄CH₃ | H | F | Cl | CF₃ | H |
| A-779 | (CH₂)₂C₆H₄CF₃ | H | F | Cl | CF₃ | H |
| A-780 | (CH₂)₂C₆H₄OCH₃ | H | F | Cl | CF₃ | H |
| A-781 | (CH₂)₂C₆H₄OCF₃ | H | F | Cl | CF₃ | H |
| A-782 | CH₂CH═CH₂ | H | F | Cl | CF₃ | H |
| A-783 | cyclo-C₃H₅ | H | F | Cl | CF₃ | H |
| A-784 | (CH₂)₂NHCH₃ | H | F | Cl | CF₃ | H |
| A-785 | H | H | Cl | H | CF₃ | 6-Cl |
| A-786 | CH₃ | CH₃ | Cl | H | CF₃ | 6-Cl |
| A-787 | CH₂CH₃ | CH₂CH₃ | Cl | H | CF₃ | 6-Cl |
| A-788 | CH₃ | H | Cl | H | CF₃ | 6-Cl |
| A-789 | CH₂CH₃ | H | Cl | H | CF₃ | 6-Cl |
| A-790 | (CH₂)₂CH₃ | H | Cl | H | CF₃ | 6-Cl |
| A-791 | CH(CH₃)₂ | H | Cl | H | CF₃ | 6-Cl |
| A-792 | CH₂CF₃ | H | Cl | H | CF₃ | 6-Cl |
| A-793 | C(CH₃)₃ | H | Cl | H | CF₃ | 6-Cl |
| A-794 | CH₂CH(CH₃)₂ | H | Cl | H | CF₃ | 6-Cl |
| A-795 | CH(CH₃)CH₂CH₃ | H | Cl | H | CF₃ | 6-Cl |
| A-796 | (CH₂)₂OCH₃ | H | Cl | H | CF₃ | 6-Cl |
| A-797 | CH₂CH(OCH₃)₂ | H | Cl | H | CF₃ | 6-Cl |
| A-798 | CH₂CH(OCH₂CH₃)₂ | H | Cl | H | CF₃ | 6-Cl |
| A-799 | CH₂C₆H₅ | H | Cl | H | CF₃ | 6-Cl |
| A-800 | CH(CH₃)C₆H₅ | H | Cl | H | CF₃ | 6-Cl |
| A-801 | (CH₂)₂OC₆H₅ | H | Cl | H | CF₃ | 6-Cl |
| A-802 | (CH₂)₂C₆H₅ | H | Cl | H | CF₃ | 6-Cl |
| A-803 | (CH₂)₂C₆H₄F | H | Cl | H | CF₃ | 6-Cl |
| A-804 | (CH₂)₂C₆H₄Cl | H | Cl | H | CF₃ | 6-Cl |
| A-805 | (CH₂)₂C₆H₄CN | H | Cl | H | CF₃ | 6-Cl |
| A-806 | (CH₂)₂C₆H₄CH₃ | H | Cl | H | CF₃ | 6-Cl |
| A-807 | (CH₂)₂C₆H₄CF₃ | H | Cl | H | CF₃ | 6-Cl |
| A-808 | (CH₂)₂C₆H₄OCH₃ | H | Cl | H | CF₃ | 6-Cl |
| A-809 | (CH₂)₂C₆H₄OCF₃ | H | Cl | H | CF₃ | 6-Cl |
| A-810 | CH₂CH═CH₂ | H | Cl | H | CF₃ | 6-Cl |
| A-811 | cyclo-C₃H₅ | H | Cl | H | CF₃ | 6-Cl |
| A-812 | (CH₂)₂NHCH₃ | H | Cl | H | CF₃ | 6-Cl |
| A-813 | H | H | Cl | H | F | 6-Cl |
| A-814 | CH₃ | CH₃ | Cl | H | F | 6-Cl |
| A-815 | CH₂CH₃ | CH₂CH₃ | Cl | H | F | 6-Cl |
| A-816 | CH₃ | H | Cl | H | F | 6-Cl |
| A-817 | CH₂CH₃ | H | Cl | H | F | 6-Cl |
| A-818 | (CH₂)₂CH₃ | H | Cl | H | F | 6-Cl |
| A-819 | CH(CH₃)₂ | H | Cl | H | F | 6-Cl |
| A-820 | CH₂CF₃ | H | Cl | H | F | 6-Cl |
| A-821 | C(CH₃)₃ | H | Cl | H | F | 6-Cl |
| A-822 | CH₂CH(CH₃)₂ | H | Cl | H | F | 6-Cl |
| A-823 | CH(CH₃)CH₂CH₃ | H | Cl | H | F | 6-Cl |
| A-824 | (CH₂)₂OCH₃ | H | Cl | H | F | 6-Cl |
| A-825 | CH₂CH(OCH₃)₂ | H | Cl | H | F | 6-Cl |
| A-826 | CH₂CH(OCH₂CH₃)₂ | H | Cl | H | F | 6-Cl |
| A-827 | CH₂C₆H₅ | H | Cl | H | F | 6-Cl |
| A-828 | CH(CH₃)C₆H₅ | H | Cl | H | F | 6-Cl |
| A-829 | (CH₂)₂OC₆H₅ | H | Cl | H | F | 6-Cl |
| A-830 | (CH₂)₂C₆H₅ | H | Cl | H | F | 6-Cl |
| A-831 | (CH₂)₂C₆H₄F | H | Cl | H | F | 6-Cl |
| A-832 | (CH₂)₂C₆H₄Cl | H | Cl | H | F | 6-Cl |
| A-833 | (CH₂)₂C₆H₄CN | H | Cl | H | F | 6-Cl |
| A-834 | (CH₂)₂C₆H₄CH₃ | H | Cl | H | F | 6-Cl |
| A-835 | (CH₂)₂C₆H₄CF₃ | H | Cl | H | F | 6-Cl |
| A-836 | (CH₂)₂C₆H₄OCH₃ | H | Cl | H | F | 6-Cl |
| A-837 | (CH₂)₂C₆H₄OCF₃ | H | Cl | H | F | 6-Cl |
| A-838 | CH₂CH═CH₂ | H | Cl | H | F | 6-Cl |
| A-839 | cyclo-C₃H₅ | H | Cl | H | F | 6-Cl |
| A-840 | (CH₂)₂NHCH₃ | H | Cl | H | F | 6-Cl |
| A-841 | H | H | F | H | Cl | 6-Cl |
| A-842 | CH₃ | CH₃ | F | H | Cl | 6-Cl |
| A-843 | CH₂CH₃ | CH₂CH₃ | F | H | Cl | 6-Cl |
| A-844 | CH₃ | H | F | H | Cl | 6-Cl |
| A-845 | CH₂CH₃ | H | F | H | Cl | 6-Cl |
| A-846 | (CH₂)₂CH₃ | H | F | H | Cl | 6-Cl |
| A-847 | CH(CH₃)₂ | H | F | H | Cl | 6-Cl |
| A-848 | CH₂CF₃ | H | F | H | Cl | 6-Cl |
| A-849 | C(CH₃)₃ | H | F | H | Cl | 6-Cl |
| A-850 | CH₂CH(CH₃)₂ | H | F | H | Cl | 6-Cl |
| A-851 | CH(CH₃)CH₂CH₃ | H | F | H | Cl | 6-Cl |
| A-852 | (CH₂)₂OCH₃ | H | F | H | Cl | 6-Cl |
| A-853 | CH₂CH(OCH₃)₂ | H | F | H | Cl | 6-Cl |
| A-854 | CH₂CH(OCH₂CH₃)₂ | H | F | H | Cl | 6-Cl |
| A-855 | CH₂C₆H₅ | H | F | H | Cl | 6-Cl |
| A-856 | CH(CH₃)C₆H₅ | H | F | H | Cl | 6-Cl |
| A-857 | (CH₂)₂OC₆H₅ | H | F | H | Cl | 6-Cl |
| A-858 | (CH₂)₂C₆H₅ | H | F | H | Cl | 6-Cl |
| A-859 | (CH₂)₂C₆H₄F | H | F | H | Cl | 6-Cl |
| A-860 | (CH₂)₂C₆H₄Cl | H | F | H | Cl | 6-Cl |
| A-861 | (CH₂)₂C₆H₄CN | H | F | H | Cl | 6-Cl |
| A-862 | (CH₂)₂C₆H₄CH₃ | H | F | H | Cl | 6-Cl |
| A-863 | (CH₂)₂C₆H₄CF₃ | H | F | H | Cl | 6-Cl |
| A-864 | (CH₂)₂C₆H₄OCH₃ | H | F | H | Cl | 6-Cl |
| A-865 | (CH₂)₂C₆H₄OCF₃ | H | F | H | Cl | 6-Cl |
| A-866 | CH₂CH═CH₂ | H | F | H | Cl | 6-Cl |
| A-867 | cyclo-C₃H₅ | H | F | H | Cl | 6-Cl |
| A-868 | (CH₂)₂NHCH₃ | H | F | H | Cl | 6-Cl |
| A-869 | H | H | Cl | H | Cl | 6-Cl |
| A-870 | CH₃ | CH₃ | Cl | H | Cl | 6-Cl |
| A-871 | CH₂CH₃ | CH₂CH₃ | Cl | H | Cl | 6-Cl |
| A-872 | CH₃ | H | Cl | H | Cl | 6-Cl |
| A-873 | CH₂CH₃ | H | Cl | H | Cl | 6-Cl |
| A-874 | (CH₂)₂CH₃ | H | Cl | H | Cl | 6-Cl |
| A-875 | CH(CH₃)₂ | H | Cl | H | Cl | 6-Cl |
| A-876 | CH₂CF₃ | H | Cl | H | Cl | 6-Cl |
| A-877 | C(CH₃)₃ | H | Cl | H | Cl | 6-Cl |
| A-878 | CH₂CH(CH₃)₂ | H | Cl | H | Cl | 6-Cl |
| A-879 | CH(CH₃)CH₂CH₃ | H | Cl | H | Cl | 6-Cl |
| A-880 | (CH₂)₂OCH₃ | H | Cl | H | Cl | 6-Cl |
| A-881 | CH₂CH(OCH₃)₂ | H | Cl | H | Cl | 6-Cl |
| A-882 | CH₂CH(OCH₂CH₃)₂ | H | Cl | H | Cl | 6-Cl |
| A-883 | CH₂C₆H₅ | H | Cl | H | Cl | 6-Cl |
| A-884 | CH(CH₃)C₆H₅ | H | Cl | H | Cl | 6-Cl |
| A-885 | (CH₂)₂OC₆H₅ | H | Cl | H | Cl | 6-Cl |
| A-886 | (CH₂)₂C₆H₅ | H | Cl | H | Cl | 6-Cl |
| A-887 | (CH₂)₂C₆H₄F | H | Cl | H | Cl | 6-Cl |
| A-888 | (CH₂)₂C₆H₄Cl | H | Cl | H | Cl | 6-Cl |
| A-889 | (CH₂)₂C₆H₄CN | H | Cl | H | Cl | 6-Cl |
| A-890 | (CH₂)₂C₆H₄CH₃ | H | Cl | H | Cl | 6-Cl |
| A-891 | (CH₂)₂C₆H₄CF₃ | H | Cl | H | Cl | 6-Cl |
| A-892 | (CH₂)₂C₆H₄OCH₃ | H | Cl | H | Cl | 6-Cl |
| A-893 | (CH₂)₂C₆H₄OCF₃ | H | Cl | H | Cl | 6-Cl |
| A-894 | CH₂CH═CH₂ | H | Cl | H | Cl | 6-Cl |
| A-895 | cyclo-C₃H₅ | H | Cl | H | Cl | 6-Cl |
| A-896 | (CH₂)₂NHCH₃ | H | Cl | H | Cl | 6-Cl |
| A-897 | H | H | F | H | Cl | 6-F |
| A-898 | CH₃ | CH₃ | F | H | Cl | 6-F |
| A-899 | CH₂CH₃ | CH₂CH₃ | F | H | Cl | 6-F |
| A-900 | CH₃ | H | F | H | Cl | 6-F |
| A-901 | CH₂CH₃ | H | F | H | Cl | 6-F |
| A-902 | (CH₂)₂CH₃ | H | F | H | Cl | 6-F |
| A-903 | CH(CH₃)₂ | H | F | H | Cl | 6-F |
| A-904 | CH₂CF₃ | H | F | H | Cl | 6-F |
| A-905 | C(CH₃)₃ | H | F | H | Cl | 6-F |
| A-906 | CH₂CH(CH₃)₂ | H | F | H | Cl | 6-F |
| A-907 | CH(CH₃)CH₂CH₃ | H | F | H | Cl | 6-F |
| A-908 | (CH₂)₂OCH₃ | H | F | H | Cl | 6-F |
| A-909 | CH₂CH(OCH₃)₂ | H | F | H | Cl | 6-F |
| A-910 | CH₂CH(OCH₂CH₃)₂ | H | F | H | Cl | 6-F |
| A-911 | CH₂C₆H₅ | H | F | H | Cl | 6-F |
| A-912 | CH(CH₃)C₆H₅ | H | F | H | Cl | 6-F |
| A-913 | (CH₂)₂OC₆H₅ | H | F | H | Cl | 6-F |
| A-914 | (CH₂)₂C₆H₅ | H | F | H | Cl | 6-F |
| A-915 | (CH₂)₂C₆H₄F | H | F | H | Cl | 6-F |
| A-916 | (CH₂)₂C₆H₄Cl | H | F | H | Cl | 6-F |
| A-917 | (CH₂)₂C₆H₄CN | H | F | H | Cl | 6-F |
| A-918 | (CH₂)₂C₆H₄CH₃ | H | F | H | Cl | 6-F |
| A-919 | (CH₂)₂C₆H₄CF₃ | H | F | H | Cl | 6-F |
| A-920 | (CH₂)₂C₆H₄OCH₃ | H | F | H | Cl | 6-F |
| A-921 | (CH₂)₂C₆H₄OCF₃ | H | F | H | Cl | 6-F |
| A-922 | CH₂CH═CH₂ | H | F | H | Cl | 6-F |
| A-923 | cyclo-C₃H₅ | H | F | H | Cl | 6-F |
| A-924 | (CH₂)₂NHCH₃ | H | F | H | Cl | 6-F |
| A-925 | H | H | F | H | F | 6-Cl |
| A-926 | CH₃ | CH₃ | F | H | F | 6-Cl |
| A-927 | CH₂CH₃ | CH₂CH₃ | F | H | F | 6-Cl |
| A-928 | CH₃ | H | F | H | F | 6-Cl |
| A-929 | CH₂CH₃ | H | F | H | F | 6-Cl |
| A-930 | (CH₂)₂CH₃ | H | F | H | F | 6-Cl |
| A-931 | CH(CH₃)₂ | H | F | H | F | 6-Cl |
| A-932 | CH₂CF₃ | H | F | H | F | 6-Cl |
| A-933 | C(CH₃)₃ | H | F | H | F | 6-Cl |
| A-934 | CH₂CH(CH₃)₂ | H | F | H | F | 6-Cl |
| A-935 | CH(CH₃)CH₂CH₃ | H | F | H | F | 6-Cl |
| A-936 | (CH₂)₂OCH₃ | H | F | H | F | 6-Cl |
| A-937 | CH₂CH(OCH₃)₂ | H | F | H | F | 6-Cl |
| A-938 | CH₂CH(OCH₂CH₃)₂ | H | F | H | F | 6-Cl |
| A-939 | CH₂C₆H₅ | H | F | H | F | 6-Cl |
| A-940 | CH(CH₃)C₆H₅ | H | F | H | F | 6-Cl |
| A-941 | (CH₂)₂OC₆H₅ | H | F | H | F | 6-Cl |
| A-942 | (CH₂)₂C₆H₅ | H | F | H | F | 6-Cl |
| A-943 | (CH₂)₂C₆H₄F | H | F | H | F | 6-Cl |
| A-944 | (CH₂)₂C₆H₄Cl | H | F | H | F | 6-Cl |
| A-945 | (CH₂)₂C₆H₄CN | H | F | H | F | 6-Cl |
| A-946 | (CH₂)₂C₆H₄CH₃ | H | F | H | F | 6-Cl |
| A-947 | (CH₂)₂C₆H₄CF₃ | H | F | H | F | 6-Cl |
| A-948 | (CH₂)₂C₆H₄OCH₃ | H | F | H | F | 6-Cl |
| A-949 | (CH₂)₂C₆H₄OCF₃ | H | F | H | F | 6-Cl |
| A-950 | CH₂CH═CH₂ | H | F | H | F | 6-Cl |
| A-951 | cyclo-C₃H₅ | H | F | H | F | 6-Cl |
| A-952 | (CH₂)₂NHCH₃ | H | F | H | F | 6-Cl |
| A-953 | H | H | F | H | CF₃ | 6-F |
| A-954 | CH₃ | CH₃ | F | H | CF₃ | 6-F |
| A-955 | CH₂CH₃ | CH₂CH₃ | F | H | CF₃ | 6-F |
| A-956 | CH₃ | H | F | H | CF₃ | 6-F |
| A-957 | CH₂CH₃ | H | F | H | CF₃ | 6-F |
| A-958 | (CH₂)₂CH₃ | H | F | H | CF₃ | 6-F |
| A-959 | CH(CH₃)₂ | H | F | H | CF₃ | 6-F |
| A-960 | CH₂CF₃ | H | F | H | CF₃ | 6-F |
| A-961 | C(CH₃)₃ | H | F | H | CF₃ | 6-F |
| A-962 | CH₂CH(CH₃)₂ | H | F | H | CF₃ | 6-F |
| A-963 | CH(CH₃)CH₂CH₃ | H | F | H | CF₃ | 6-F |
| A-964 | (CH₂)₂OCH₃ | H | F | H | CF₃ | 6-F |
| A-965 | CH₂CH(OCH₃)₂ | H | F | H | CF₃ | 6-F |
| A-966 | CH₂CH(OCH₂CH₃)₂ | H | F | H | CF₃ | 6-F |
| A-967 | CH₂C₆H₅ | H | F | H | CF₃ | 6-F |
| A-968 | CH(CH₃)C₆H₅ | H | F | H | CF₃ | 6-F |
| A-969 | (CH₂)₂OC₆H₅ | H | F | H | CF₃ | 6-F |
| A-970 | (CH₂)₂C₆H₅ | H | F | H | CF₃ | 6-F |
| A-971 | (CH₂)₂C₆H₄F | H | F | H | CF₃ | 6-F |
| A-972 | (CH₂)₂C₆H₄Cl | H | F | H | CF₃ | 6-F |
| A-973 | (CH₂)₂C₆H₄CN | H | F | H | CF₃ | 6-F |
| A-974 | (CH₂)₂C₆H₄CH₃ | H | F | H | CF₃ | 6-F |
| A-975 | (CH₂)₂C₆H₄CF₃ | H | F | H | CF₃ | 6-F |
| A-976 | (CH₂)₂C₆H₄OCH₃ | H | F | H | CF₃ | 6-F |
| A-977 | (CH₂)₂C₆H₄OCF₃ | H | F | H | CF₃ | 6-F |
| A-978 | CH₂CH═CH₂ | H | F | H | CF₃ | 6-F |
| A-979 | cyclo-C₃H₅ | H | F | H | CF₃ | 6-F |
| A-980 | (CH₂)₂NHCH₃ | H | F | H | CF₃ | 6-F |
| A-981 | H | H | CF₃ | H | F | 6-F |
| A-982 | CH₃ | CH₃ | CF₃ | H | F | 6-F |
| A-983 | CH₂CH₃ | CH₂CH₃ | CF₃ | H | F | 6-F |
| A-984 | CH₃ | H | CF₃ | H | F | 6-F |
| A-985 | CH₂CH₃ | H | CF₃ | H | F | 6-F |
| A-986 | (CH₂)₂CH₃ | H | CF₃ | H | F | 6-F |
| A-987 | CH(CH₃)₂ | H | CF₃ | H | F | 6-F |
| A-988 | CH₂CF₃ | H | CF₃ | H | F | 6-F |
| A-989 | C(CH₃)₃ | H | CF₃ | H | F | 6-F |
| A-990 | CH₂CH(CH₃)₂ | H | CF₃ | H | F | 6-F |
| A-991 | CH(CH₃)CH₂CH₃ | H | CF₃ | H | F | 6-F |
| A-992 | (CH₂)₂OCH₃ | H | CF₃ | H | F | 6-F |
| A-993 | CH₂CH(OCH₃)₂ | H | CF₃ | H | F | 6-F |
| A-994 | CH₂CH(OCH₂CH₃)₂ | H | CF₃ | H | F | 6-F |
| A-995 | CH₂C₆H₅ | H | CF₃ | H | F | 6-F |
| A-996 | CH(CH₃)C₆H₅ | H | CF₃ | H | F | 6-F |
| A-997 | (CH₂)₂OC₆H₅ | H | CF₃ | H | F | 6-F |
| A-998 | (CH₂)₂C₆H₅ | H | CF₃ | H | F | 6-F |
| A-999 | (CH₂)₂C₆H₄F | H | CF₃ | H | F | 6-F |
| A-1000 | (CH₂)₂C₆H₄Cl | H | CF₃ | H | F | 6-F |
| A-1001 | (CH₂)₂C₆H₄CN | H | CF₃ | H | F | 6-F |
| A-1002 | (CH₂)₂C₆H₄CH₃ | H | CF₃ | H | F | 6-F |
| A-1003 | (CH₂)₂C₆H₄CF₃ | H | CF₃ | H | F | 6-F |
| A-1004 | (CH₂)₂C₆H₄OCH₃ | H | CF₃ | H | F | 6-F |
| A-1005 | (CH₂)₂C₆H₄OCF₃ | H | CF₃ | H | F | 6-F |
| A-1006 | CH₂CH═CH₂ | H | CF₃ | H | F | 6-F |
| A-1007 | cyclo-C₃H₅ | H | CF₃ | H | F | 6-F |
| A-1008 | (CH₂)₂NHCH₃ | H | CF₃ | H | F | 6-F |
| A-1009 | H | H | CF₃ | H | F | 6-CF₃ |
| A-1010 | CH₃ | CH₃ | CF₃ | H | F | 6-CF₃ |
| A-1011 | CH₂CH₃ | CH₂CH₃ | CF₃ | H | F | 6-CF₃ |
| A-1012 | CH₃ | H | CF₃ | H | F | 6-CF₃ |
| A-1013 | CH₂CH₃ | H | CF₃ | H | F | 6-CF₃ |
| A-1014 | (CH₂)₂CH₃ | H | CF₃ | H | F | 6-CF₃ |
| A-1015 | CH(CH₃)₂ | H | CF₃ | H | F | 6-CF₃ |
| A-1016 | CH₂CF₃ | H | CF₃ | H | F | 6-CF₃ |
| A-1017 | C(CH₃)₃ | H | CF₃ | H | F | 6-CF₃ |
| A-1018 | CH₂CH(CH₃)₂ | H | CF₃ | H | F | 6-CF₃ |
| A-1019 | CH(CH₃)CH₂CH₃ | H | CF₃ | H | F | 6-CF₃ |
| A-1020 | (CH₂)₂OCH₃ | H | CF₃ | H | F | 6-CF₃ |
| A-1021 | CH₂CH(OCH₃)₂ | H | CF₃ | H | F | 6-CF₃ |
| A-1022 | CH₂CH(OCH₂CH₃)₂ | H | CF₃ | H | F | 6-CF₃ |
| A-1023 | CH₂C₆H₅ | H | CF₃ | H | F | 6-CF₃ |
| A-1024 | CH(CH₃)C₆H₅ | H | CF₃ | H | F | 6-CF₃ |
| A-1025 | (CH₂)₂OC₆H₅ | H | CF₃ | H | F | 6-CF₃ |
| A-1026 | (CH₂)₂C₆H₅ | H | CF₃ | H | F | 6-CF₃ |
| A-1027 | (CH₂)₂C₆H₄F | H | CF₃ | H | F | 6-CF₃ |
| A-1028 | (CH₂)₂C₆H₄Cl | H | CF₃ | H | F | 6-CF₃ |
| A-1029 | (CH₂)₂C₆H₄CN | H | CF₃ | H | F | 6-CF₃ |
| A-1030 | (CH₂)₂C₆H₄CH₃ | H | CF₃ | H | F | 6-CF₃ |
| A-1031 | (CH₂)₂C₆H₄CF₃ | H | CF₃ | H | F | 6-CF₃ |
| A-1032 | (CH₂)₂C₆H₄OCH₃ | H | CF₃ | H | F | 6-CF₃ |
| A-1033 | (CH₂)₂C₆H₄OCF₃ | H | CF₃ | H | F | 6-CF₃ |
| A-1034 | CH₂CH═CH₂ | H | CF₃ | H | F | 6-CF₃ |
| A-1035 | cyclo-C₃H₅ | H | CF₃ | H | F | 6-CF₃ |
| A-1036 | (CH₂)₂NHCH₃ | H | CF₃ | H | F | 6-CF₃ |
| A-1037 | H | H | CF₃ | H | CF₃ | 6-F |
| A-1038 | CH₃ | CH₃ | CF₃ | H | CF₃ | 6-F |
| A-1039 | CH₂CH₃ | CH₂CH₃ | CF₃ | H | CF₃ | 6-F |
| A-1040 | CH₃ | H | CF₃ | H | CF₃ | 6-F |
| A-1041 | CH₂CH₃ | H | CF₃ | H | CF₃ | 6-F |
| A-1042 | (CH₂)₂CH₃ | H | CF₃ | H | CF₃ | 6-F |
| A-1043 | CH(CH₃)₂ | H | CF₃ | H | CF₃ | 6-F |
| A-1044 | CH₂CF₃ | H | CF₃ | H | CF₃ | 6-F |
| A-1045 | C(CH₃)₃ | H | CF₃ | H | CF₃ | 6-F |
| A-1046 | CH₂CH(CH₃)₂ | H | CF₃ | H | CF₃ | 6-F |
| A-1047 | CH(CH₃)CH₂CH₃ | H | CF₃ | H | CF₃ | 6-F |
| A-1048 | (CH₂)₂OCH₃ | H | CF₃ | H | CF₃ | 6-F |
| A-1049 | CH₂CH(OCH₃)₂ | H | CF₃ | H | CF₃ | 6-F |
| A-1050 | CH₂CH(OCH₂CH₃)₂ | H | CF₃ | H | CF₃ | 6-F |
| A-1051 | CH₂C₆H₅ | H | CF₃ | H | CF₃ | 6-F |
| A-1052 | CH(CH₃)C₆H₅ | H | CF₃ | H | CF₃ | 6-F |
| A-1053 | (CH₂)₂OC₆H₅ | H | CF₃ | H | CF₃ | 6-F |
| A-1054 | (CH₂)₂C₆H₅ | H | CF₃ | H | CF₃ | 6-F |
| A-1055 | (CH₂)₂C₆H₄F | H | CF₃ | H | CF₃ | 6-F |
| A-1056 | (CH₂)₂C₆H₄Cl | H | CF₃ | H | CF₃ | 6-F |
| A-1057 | (CH₂)₂C₆H₄CN | H | CF₃ | H | CF₃ | 6-F |
| A-1058 | (CH₂)₂C₆H₄CH₃ | H | CF₃ | H | CF₃ | 6-F |
| A-1059 | (CH₂)₂C₆H₄CF₃ | H | CF₃ | H | CF₃ | 6-F |
| A-1060 | (CH₂)₂C₆H₄OCH₃ | H | CF₃ | H | CF₃ | 6-F |
| A-1061 | (CH₂)₂C₆H₄OCF₃ | H | CF₃ | H | CF₃ | 6-F |
| A-1062 | CH₂CH═CH₂ | H | CF₃ | H | CF₃ | 6-F |
| A-1063 | cyclo-C₃H₅ | H | CF₃ | H | CF₃ | 6-F |
| A-1064 | (CH₂)₂NHCH₃ | H | CF₃ | H | CF₃ | 6-F |
| A-1065 | H | H | CF₃ | H | CF₃ | 6-Cl |
| A-1066 | CH₃ | CH₃ | CF₃ | H | CF₃ | 6-Cl |
| A-1067 | CH₂CH₃ | CH₂CH₃ | CF₃ | H | CF₃ | 6-Cl |
| A-1068 | CH₃ | H | CF₃ | H | CF₃ | 6-Cl |
| A-1069 | CH₂CH₃ | H | CF₃ | H | CF₃ | 6-Cl |
| A-1070 | (CH₂)₂CH₃ | H | CF₃ | H | CF₃ | 6-Cl |
| A-1071 | CH(CH₃)₂ | H | CF₃ | H | CF₃ | 6-Cl |
| A-1072 | CH₂CF₃ | H | CF₃ | H | CF₃ | 6-Cl |
| A-1073 | C(CH₃)₃ | H | CF₃ | H | CF₃ | 6-Cl |
| A-1074 | CH₂CH(CH₃)₂ | H | CF₃ | H | CF₃ | 6-Cl |
| A-1075 | CH(CH₃)CH₂CH₃ | H | CF₃ | H | CF₃ | 6-Cl |
| A-1076 | (CH₂)₂OCH₃ | H | CF₃ | H | CF₃ | 6-Cl |
| A-1077 | CH₂CH(OCH₃)₂ | H | CF₃ | H | CF₃ | 6-Cl |
| A-1078 | CH₂CH(OCH₂CH₃)₂ | H | CF₃ | H | CF₃ | 6-Cl |
| A-1079 | CH₂C₆H₅ | H | CF₃ | H | CF₃ | 6-Cl |
| A-1080 | CH(CH₃)C₆H₅ | H | CF₃ | H | CF₃ | 6-Cl |
| A-1081 | (CH₂)₂OC₆H₅ | H | CF₃ | H | CF₃ | 6-Cl |
| A-1082 | (CH₂)₂C₆H₅ | H | CF₃ | H | CF₃ | 6-Cl |
| A-1083 | (CH₂)₂C₆H₄F | H | CF₃ | H | CF₃ | 6-Cl |
| A-1084 | (CH₂)₂C₆H₄Cl | H | CF₃ | H | CF₃ | 6-Cl |
| A-1085 | (CH₂)₂C₆H₄CN | H | CF₃ | H | CF₃ | 6-Cl |
| A-1086 | (CH₂)₂C₆H₄CH₃ | H | CF₃ | H | CF₃ | 6-Cl |
| A-1087 | (CH₂)₂C₆H₄CF₃ | H | CF₃ | H | CF₃ | 6-Cl |
| A-1088 | (CH₂)₂C₆H₄OCH₃ | H | CF₃ | H | CF₃ | 6-Cl |
| A-1089 | (CH₂)₂C₆H₄OCF₃ | H | CF₃ | H | CF₃ | 6-Cl |
| A-1090 | CH₂CH═CH₂ | H | CF₃ | H | CF₃ | 6-Cl |
| A-1091 | cyclo-C₃H₅ | H | CF₃ | H | CF₃ | 6-Cl |
| A-1092 | (CH₂)₂NHCH₃ | H | CF₃ | H | CF₃ | 6-Cl |
| A-1093 | H | H | CF₃ | H | Cl | 6-Cl |
| A-1094 | CH₃ | CH₃ | CF₃ | H | Cl | 6-Cl |
| A-1095 | CH₂CH₃ | CH₂CH₃ | CF₃ | H | Cl | 6-Cl |
| A-1096 | CH₃ | H | CF₃ | H | Cl | 6-Cl |
| A-1097 | CH₂CH₃ | H | CF₃ | H | Cl | 6-Cl |
| A-1098 | (CH₂)₂CH₃ | H | CF₃ | H | Cl | 6-Cl |
| A-1099 | CH(CH₃)₂ | H | CF₃ | H | Cl | 6-Cl |
| A-1100 | CH₂CF₃ | H | CF₃ | H | Cl | 6-Cl |
| A-1101 | C(CH₃)₃ | H | CF₃ | H | Cl | 6-Cl |
| A-1102 | CH₂CH(CH₃)₂ | H | CF₃ | H | Cl | 6-Cl |
| A-1103 | CH(CH₃)CH₂CH₃ | H | CF₃ | H | Cl | 6-Cl |
| A-1104 | (CH₂)₂OCH₃ | H | CF₃ | H | Cl | 6-Cl |
| A-1105 | CH₂CH(OCH₃)₂ | H | CF₃ | H | Cl | 6-Cl |
| A-1106 | CH₂CH(OCH₂CH₃)₂ | H | CF₃ | H | Cl | 6-Cl |
| A-1107 | CH₂C₆H₅ | H | CF₃ | H | Cl | 6-Cl |
| A-1108 | CH(CH₃)C₆H₅ | H | CF₃ | H | Cl | 6-Cl |
| A-1109 | (CH₂)₂OC₆H₅ | H | CF₃ | H | Cl | 6-Cl |
| A-1110 | (CH₂)₂C₆H₅ | H | CF₃ | H | Cl | 6-Cl |
| A-1111 | (CH₂)₂C₆H₄F | H | CF₃ | H | Cl | 6-Cl |
| A-1112 | (CH₂)₂C₆H₄Cl | H | CF₃ | H | Cl | 6-Cl |
| A-1113 | (CH₂)₂C₆H₄CN | H | CF₃ | H | Cl | 6-Cl |
| A-1114 | (CH₂)₂C₆H₄CH₃ | H | CF₃ | H | Cl | 6-Cl |
| A-1115 | (CH₂)₂C₆H₄CF₃ | H | CF₃ | H | Cl | 6-Cl |
| A-1116 | (CH₂)₂C₆H₄OCH₃ | H | CF₃ | H | Cl | 6-Cl |
| A-1117 | (CH₂)₂C₆H₄OCF₃ | H | CF₃ | H | Cl | 6-Cl |
| A-1118 | CH₂CH═CH₂ | H | CF₃ | H | Cl | 6-Cl |
| A-1119 | cyclo-C₃H₅ | H | CF₃ | H | Cl | 6-Cl |
| A-1120 | (CH₂)₂NHCH₃ | H | CF₃ | H | Cl | 6-Cl |
| A-1121 | H | H | CF₃ | H | Cl | 6-CF₃ |
| A-1122 | CH₃ | CH₃ | CF₃ | H | Cl | 6-CF₃ |
| A-1123 | CH₂CH₃ | CH₂CH₃ | CF₃ | H | Cl | 6-CF₃ |
| A-1124 | CH₃ | H | CF₃ | H | Cl | 6-CF₃ |
| A-1125 | CH₂CH₃ | H | CF₃ | H | Cl | 6-CF₃ |
| A-1126 | (CH₂)₂CH₃ | H | CF₃ | H | Cl | 6-CF₃ |
| A-1127 | CH(CH₃)₂ | H | CF₃ | H | Cl | 6-CF₃ |
| A-1128 | CH₂CF₃ | H | CF₃ | H | Cl | 6-CF₃ |
| A-1129 | C(CH₃)₃ | H | CF₃ | H | Cl | 6-CF₃ |
| A-1130 | CH₂CH(CH₃)₂ | H | CF₃ | H | Cl | 6-CF₃ |
| A-1131 | CH(CH₃)CH₂CH₃ | H | CF₃ | H | Cl | 6-CF₃ |
| A-1132 | (CH₂)₂OCH₃ | H | CF₃ | H | Cl | 6-CF₃ |
| A-1133 | CH₂CH(OCH₃)₂ | H | CF₃ | H | Cl | 6-CF₃ |
| A-1134 | CH₂CH(OCH₂CH₃)₂ | H | CF₃ | H | Cl | 6-CF₃ |
| A-1135 | CH₂C₆H₅ | H | CF₃ | H | Cl | 6-CF₃ |
| A-1136 | CH(CH₃)C₆H₅ | H | CF₃ | H | Cl | 6-CF₃ |
| A-1137 | (CH₂)₂OC₆H₅ | H | CF₃ | H | Cl | 6-CF₃ |
| A-1138 | (CH₂)₂C₆H₅ | H | CF₃ | H | Cl | 6-CF₃ |
| A-1139 | (CH₂)₂C₆H₄F | H | CF₃ | H | Cl | 6-CF₃ |
| A-1140 | (CH₂)₂C₆H₄Cl | H | CF₃ | H | Cl | 6-CF₃ |
| A-1141 | (CH₂)₂C₆H₄CN | H | CF₃ | H | Cl | 6-CF₃ |
| A-1142 | (CH₂)₂C₆H₄CH₃ | H | CF₃ | H | Cl | 6-CF₃ |
| A-1143 | (CH₂)₂C₆H₄CF₃ | H | CF₃ | H | Cl | 6-CF₃ |
| A-1144 | (CH₂)₂C₆H₄OCH₃ | H | CF₃ | H | Cl | 6-CF₃ |
| A-1145 | (CH₂)₂C₆H₄OCF₃ | H | CF₃ | H | Cl | 6-CF₃ |
| A-1146 | CH₂CH═CH₂ | H | CF₃ | H | Cl | 6-CF₃ |
| A-1147 | cyclo-C₃H₅ | H | CF₃ | H | Cl | 6-CF₃ |
| A-1148 | (CH₂)₂NHCH₃ | H | CF₃ | H | Cl | 6-CF₃ |
| A-1149 | H | H | F | H | CF₃ | 6-Cl |
| A-1150 | CH₃ | CH₃ | F | H | CF₃ | 6-Cl |
| A-1151 | CH₂CH₃ | CH₂CH₃ | F | H | CF₃ | 6-Cl |
| A-1152 | CH₃ | H | F | H | CF₃ | 6-Cl |
| A-1153 | CH₂CH₃ | H | F | H | CF₃ | 6-Cl |
| A-1154 | (CH₂)₂CH₃ | H | F | H | CF₃ | 6-Cl |
| A-1155 | CH(CH₃)₂ | H | F | H | CF₃ | 6-Cl |
| A-1156 | CH₂CF₃ | H | F | H | CF₃ | 6-Cl |
| A-1157 | C(CH₃)₃ | H | F | H | CF₃ | 6-Cl |
| A-1158 | CH₂CH(CH₃)₂ | H | F | H | CF₃ | 6-Cl |
| A-1159 | CH(CH₃)CH₂CH₃ | H | F | H | CF₃ | 6-Cl |
| A-1160 | (CH₂)₂OCH₃ | H | F | H | CF₃ | 6-Cl |
| A-1161 | CH₂CH(OCH₃)₂ | H | F | H | CF₃ | 6-Cl |
| A-1162 | CH₂CH(OCH₂CH₃)₂ | H | F | H | CF₃ | 6-Cl |
| A-1163 | CH₂C₆H₅ | H | F | H | CF₃ | 6-Cl |
| A-1164 | CH(CH₃)C₆H₅ | H | F | H | CF₃ | 6-Cl |
| A-1165 | (CH₂)₂OC₆H₅ | H | F | H | CF₃ | 6-Cl |
| A-1166 | (CH₂)₂C₆H₅ | H | F | H | CF₃ | 6-Cl |
| A-1167 | (CH₂)₂C₆H₄F | H | F | H | CF₃ | 6-Cl |
| A-1168 | (CH₂)₂C₆H₄Cl | H | F | H | CF₃ | 6-Cl |
| A-1169 | (CH₂)₂C₆H₄CN | H | F | H | CF₃ | 6-Cl |
| A-1170 | (CH₂)₂C₆H₄CH₃ | H | F | H | CF₃ | 6-Cl |
| A-1171 | (CH₂)₂C₆H₄CF₃ | H | F | H | CF₃ | 6-Cl |
| A-1172 | (CH₂)₂C₆H₄OCH₃ | H | F | H | CF₃ | 6-Cl |
| A-1173 | (CH₂)₂C₆H₄OCF₃ | H | F | H | CF₃ | 6-Cl |
| A-1174 | CH₂CH═CH₂ | H | F | H | CF₃ | 6-Cl |
| A-1175 | cyclo-C₃H₅ | H | F | H | CF₃ | 6-Cl |
| A-1176 | (CH₂)₂NHCH₃ | H | F | H | CF₃ | 6-Cl |
| A-1177 | H | H | F | H | F | 6-F |
| A-1178 | CH₃ | CH₃ | F | H | F | 6-F |
| A-1179 | CH₂CH₃ | CH₂CH₃ | F | H | F | 6-F |
| A-1180 | CH₃ | H | F | H | F | 6-F |
| A-1181 | CH₂CH₃ | H | F | H | F | 6-F |
| A-1182 | (CH₂)₂CH₃ | H | F | H | F | 6-F |
| A-1183 | CH(CH₃)₂ | H | F | H | F | 6-F |
| A-1184 | CH₂CF₃ | H | F | H | F | 6-F |
| A-1185 | C(CH₃)₃ | H | F | H | F | 6-F |
| A-1186 | CH₂CH(CH₃)₂ | H | F | H | F | 6-F |
| A-1187 | CH(CH₃)CH₂CH₃ | H | F | H | F | 6-F |
| A-1188 | (CH₂)₂OCH₃ | H | F | H | F | 6-F |
| A-1189 | CH₂CH(OCH₃)₂ | H | F | H | F | 6-F |
| A-1190 | CH₂CH(OCH₂CH₃)₂ | H | F | H | F | 6-F |
| A-1191 | CH₂C₆H₅ | H | F | H | F | 6-F |
| A-1192 | CH(CH₃)C₆H₅ | H | F | H | F | 6-F |
| A-1193 | (CH₂)₂OC₆H₅ | H | F | H | F | 6-F |
| A-1194 | (CH₂)₂C₆H₅ | H | F | H | F | 6-F |
| A-1195 | (CH₂)₂C₆H₄F | H | F | H | F | 6-F |
| A-1196 | (CH₂)₂C₆H₄Cl | H | F | H | F | 6-F |
| A-1197 | (CH₂)₂C₆H₄CN | H | F | H | F | 6-F |
| A-1198 | (CH₂)₂C₆H₄CH₃ | H | F | H | F | 6-F |
| A-1199 | (CH₂)₂C₆H₄CF₃ | H | F | H | F | 6-F |
| A-1200 | (CH₂)₂C₆H₄OCH₃ | H | F | H | F | 6-F |
| A-1201 | (CH₂)₂C₆H₄OCF₃ | H | F | H | F | 6-F |
| A-1202 | CH₂CH═CH₂ | H | F | H | F | 6-F |
| A-1203 | cyclo-C₃H₅ | H | F | H | F | 6-F |
| A-1204 | (CH₂)₂NHCH₃ | H | F | H | F | 6-F |
| A-1205 | H | H | Cl | Cl | Cl | 5,6-Cl₂ |
| A-1206 | CH₃ | CH₃ | Cl | Cl | Cl | 5,6-Cl₂ |
| A-1207 | CH₂CH₃ | CH₂CH₃ | Cl | Cl | Cl | 5,6-Cl₂ |
| A-1208 | CH₃ | H | Cl | Cl | Cl | 5,6-Cl₂ |
| A-1209 | CH₂CH₃ | H | Cl | Cl | Cl | 5,6-Cl₂ |
| A-1210 | (CH₂)₂CH₃ | H | Cl | Cl | Cl | 5,6-Cl₂ |
| A-1211 | CH(CH₃)₂ | H | Cl | Cl | Cl | 5,6-Cl₂ |
| A-1212 | CH₂CF₃ | H | Cl | Cl | Cl | 5,6-Cl₂ |
| A-1213 | C(CH₃)₃ | H | Cl | Cl | Cl | 5,6-Cl₂ |
| A-1214 | CH₂CH(CH₃)₂ | H | Cl | Cl | Cl | 5,6-Cl₂ |
| A-1215 | CH(CH₃)CH₂CH₃ | H | Cl | Cl | Cl | 5,6-Cl₂ |
| A-1216 | (CH₂)₂OCH₃ | H | Cl | Cl | Cl | 5,6-Cl₂ |
| A-1217 | CH₂CH(OCH₃)₂ | H | Cl | Cl | Cl | 5,6-Cl₂ |
| A-1218 | CH₂CH(OCH₂CH₃)₂ | H | Cl | Cl | Cl | 5,6-Cl₂ |
| A-1219 | CH₂C₆H₅ | H | Cl | Cl | Cl | 5,6-Cl₂ |
| A-1220 | CH(CH₃)C₆H₅ | H | Cl | Cl | Cl | 5,6-Cl₂ |
| A-1221 | (CH₂)₂OC₆H₅ | H | Cl | Cl | Cl | 5,6-Cl₂ |
| A-1222 | (CH₂)₂C₆H₅ | H | Cl | Cl | Cl | 5,6-Cl₂ |
| A-1223 | (CH₂)₂C₆H₄F | H | Cl | Cl | Cl | 5,6-Cl₂ |
| A-1224 | (CH₂)₂C₆H₄Cl | H | Cl | Cl | Cl | 5,6-Cl₂ |
| A-1225 | (CH₂)₂C₆H₄CN | H | Cl | Cl | Cl | 5,6-Cl₂ |
| A-1226 | (CH₂)₂C₆H₄CH₃ | H | Cl | Cl | Cl | 5,6-Cl₂ |
| A-1227 | (CH₂)₂C₆H₄CF₃ | H | Cl | Cl | Cl | 5,6-Cl₂ |
| A-1228 | (CH₂)₂C₆H₄OCH₃ | H | Cl | Cl | Cl | 5,6-Cl₂ |
| A-1229 | (CH₂)₂C₆H₄OCF₃ | H | Cl | Cl | Cl | 5,6-Cl₂ |
| A-1230 | CH₂CH═CH₂ | H | Cl | Cl | Cl | 5,6-Cl₂ |
| A-1231 | cyclo-C₃H₅ | H | Cl | Cl | Cl | 5,6-Cl₂ |
| A-1232 | (CH₂)₂NHCH₃ | H | Cl | Cl | Cl | 5,6-Cl₂ |
| A-1233 | H | H | F | F | F | 5,6-F₂ |
| A-1234 | CH₃ | CH₃ | F | F | F | 5,6-F₂ |
| A-1235 | CH₂CH₃ | CH₂CH₃ | F | F | F | 5,6-F₂ |
| A-1236 | CH₃ | H | F | F | F | 5,6-F₂ |
| A-1237 | CH₂CH₃ | H | F | F | F | 5,6-F₂ |
| A-1238 | (CH₂)₂CH₃ | H | F | F | F | 5,6-F₂ |
| A-1239 | CH(CH₃)₂ | H | F | F | F | 5,6-F₂ |
| A-1240 | CH₂CF₃ | H | F | F | F | 5,6-F₂ |
| A-1241 | C(CH₃)₃ | H | F | F | F | 5,6-F₂ |
| A-1242 | CH₂CH(CH₃)₂ | H | F | F | F | 5,6-F₂ |
| A-1243 | CH(CH₃)CH₂CH₃ | H | F | F | F | 5,6-F₂ |
| A-1244 | (CH₂)₂OCH₃ | H | F | F | F | 5,6-F₂ |
| A-1245 | CH₂CH(OCH₃)₂ | H | F | F | F | 5,6-F₂ |
| A-1246 | CH₂CH(OCH₂CH₃)₂ | H | F | F | F | 5,6-F₂ |
| A-1247 | CH₂C₆H₅ | H | F | F | F | 5,6-F₂ |
| A-1248 | CH(CH₃)C₆H₅ | H | F | F | F | 5,6-F₂ |
| A-1249 | (CH₂)₂OC₆H₅ | H | F | F | F | 5,6-F₂ |
| A-1250 | (CH₂)₂C₆H₅ | H | F | F | F | 5,6-F₂ |
| A-1251 | (CH₂)₂C₆H₄F | H | F | F | F | 5,6-F₂ |
| A-1252 | (CH₂)₂C₆H₄Cl | H | F | F | F | 5,6-F₂ |
| A-1253 | (CH₂)₂C₆H₄CN | H | F | F | F | 5,6-F₂ |
| A-1254 | (CH₂)₂C₆H₄CH₃ | H | F | F | F | 5,6-F₂ |
| A-1255 | (CH₂)₂C₆H₄CF₃ | H | F | F | F | 5,6-F₂ |
| A-1256 | (CH₂)₂C₆H₄OCH₃ | H | F | F | F | 5,6-F₂ |
| A-1257 | (CH₂)₂C₆H₄OCF₃ | H | F | F | F | 5,6-F₂ |
| A-1258 | CH₂CH═CH₂ | H | F | F | F | 5,6-F₂ |
| A-1259 | cyclo-C₃H₅ | H | F | F | F | 5,6-F₂ |
| A-1260 | (CH₂)₂NHCH₃ | H | F | F | F | 5,6-F₂ |

Particularly preferred with respect to the intended use in the present invention are compounds of the formula I-B. 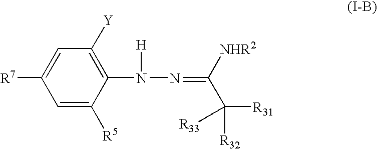
wherein
R⁷ is chlorine or trifluoromethyl;
R⁵ and Y are each independently chlorine or bromine;
R² is C₁-C₆-alkyl, C₃-C₆-alkenyl, C₃-C₆-alkynyl, or
   C₃-C₆-cycloalkyl which may be substituted with 1 to 3 halogen atoms, or
   C₂-C₄-alkyl which is substituted by C₁-C₄-alkoxy;
R³¹ and R³² are C₁-C₆-alkyl or may be taken together to form C₃-C₆-cycloalkyl which may be unsubstituted or substituted by 1 to 3 halogen atoms;
R³³ is hydrogen or C₁-C₆-alkyl,
   or the enantiomers or veterinarily acceptable salts thereof.

Preference is given to compounds of formula I-B wherein R⁷ is trifluoromethyl.

Preference is further given to compounds of formula I-B wherein Y and R⁵ are both chlorine.

Moreover, preferred are compounds of formula I-B wherein R² is C₁-C₆-alkyl, especially ethyl.

Preference is further given to compounds of formula I-B wherein R³¹ and R³² are both methyl.

Moreover, preferred are compounds of formula I-B wherein R³¹ and R³² form a cyclopropyl ring which is unsubstituted or substituted by 1 to 3 halogen atoms, especially chlorine and bromine.

Moreover, particularly preferred are compounds of formula I-B wherein R³¹ and R³² form a cyclopropyl ring which is substituted by 2 halogen atoms.

Moreover, particularly preferred are compounds of formula I-B wherein R³¹ and R³² form a cyclopropyl ring which is substituted by 2 chlorine atoms.

Particularly preferred are compounds of formula I-B wherein R³¹ and R³² form a 2,2-dichlorocyclopropyl ring.

Preference is further given to compounds of formula I-B wherein R³³ is C₁-C₆-alkyl, especially methyl.

Particularly preferred are compounds of formula I-B wherein R³¹, R³² and R³³ are all methyl.

Moreover, particularly preferred are compounds of formula I-B wherein R³¹, R³² and R³³ form a moiety 1-methyl-2,2-dichlorocyclopropyl.

Preference is further given to compounds of formula I-B wherein

R⁷ is trifluoromethyl;

Y and R⁵ are each independently chlorine or bromine;

R² is C₁-C₆alkyl;

R³¹ and R³² are C₁-C₆-alkyl or may be taken together to form C₃-C₆-cycloalkyl which is substituted by 1 to 2 halogen atoms;

R³³ is C₁-C₆-alkyl;

or the enantiomers or veterinarily acceptable salts thereof.

Particular preference is given to N-ethyl-2,2-dimethylpropionamide-2-(2,6-dichloro-α,α,α-trifluoro-p-tolyl)hydrazone and N-Ethyl-2,2-dichloro-1-methylcyclopropane-carboxamide-2-(2,6-dichloro-α,α,α-trifluoro-p-tolyl)hydrazone.

Furthermore, particular preference with respect to the use in the present invention is given to the compound of formula I-1 (N-ethyl-2,2-dimethylpropionamide-2-(2,6-dichloro-α,α,α-trifluoro-p-tolyl)-hydrazone):

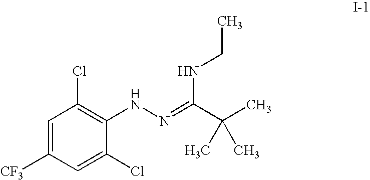

Moreover, particular preference with respect to the use in the present invention is given to the compound of formula I-2 (N-Ethyl-2,2-dichloro-1-methylcyclopropane-carboxamide-2-(2,6-dichloro-α,α,α-trifluoro-p-tolyl)hydrazone):

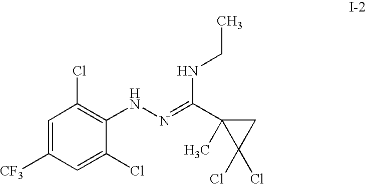

Compounds of formula I and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Amblycerina and Ischnocerina), e.g. Trimenopon spp., Menopon spp., Trinoton spp., Bovicola spp., Werneckiella spp., Lepikentron spp., Trichodectes spp., and Felicola spp,

Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworn), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

Planarians (Plathelminthes):

Flukes (Trematoda), e.g. Faciola spp., Fascioloides magna, Paragonimus. spp., Dicrocoelium spp., Fasciolopsis buski, Clonorchis sinensis, Schistosoma spp., Trichobilharzia spp., Alada alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of the compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:

Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:
liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids,
fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter,
fatty alcohols such as isotridecyl alcohol, 2-octyldodecanol, cetylstearyl alcohol, oleyl alcohol, and
fatty acids such as oleic acid and
mixtures thereof.

Suitable hydrophilic phases are: water, alcohols such as propylene glycol, glycerol, sorbitol and mixtures thereof.

Suitable emulsifiers are:
non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin;
anionic surfactants, such as sodium lauryl sulfate, fatty alcohol ether sulfates, mono/dialkyl polyglycol ether orthophosphoric acid ester monoethanolamine salt;
cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

The active compounds can also be used as a mixture with synergists or with other active compounds which act against pathogenic endo- and ectoparasites.

The following list of pesticides together with which the compounds according to the invention can be used, is intended to illustrate the possible combinations, but not to impose any limitation:

Organophosphates: Acephate, Chlorfenvinphos, Diazinon, Dichlorvos, Dicrotophos, Dimethoate, Ethion, Fenitrothion, Fenthion, Isoxathion, Malathion, Phenthoate, Phosalone, Phosmet, Phoxim, Pirimiphos-methyl, Profenofos, Prothiofos, Sulprophos, Triazophos, Trichlorfon, Quintiofos, Coumaphos, Chlorphoxim, Bromophos-ethyl, 2,3-p-Dioxanedithiol-S,S-bis(O,O-diethylphosphorodithionat);

Carbamates: Alanycarb, Benfuracarb, Carbaryl, Carbosulfan, Fenoxycarb, Furathiocarb, Indoxacarb, Triazamate;

Pyrethroids: alpha-Cypermethrin, Deltamethrin, Ethofenprox, Fenvalerate, Cyhalothrin, Lambda-Cyhalothrin, Permethrin, Silafluofen, Tau-Fluvalinate, Tralomethrin, Zeta-Cypermethrin, Flumethrin, Cyfluthrin and its enantiomers and stereomers, Cypermethrin;

Arthropod growth regulators: a) chitin synthesis inhibitors: benzoylureas: Chlorfluazuron, Cyromazine, Diflubenzuron, Flucycloxuron, Flufenoxuron, Hexaflumuron, Lufenuron, Novaluron, Teflubenzuron, Triflumuron; Buprofezin, Diofenolan, Hexythiazox, Etoxazole, Clofentazine; b) ecdysone antagonists: Halofenozide, Methoxyfenozide, Tebufenozide; c) juvenoids: Pyriproxyfen, Methoprene, Fenoxycarb; d) lipid biosynthesis inhibitors: Spirodiclofen;

Neonicotinoids: Acetamiprid, Clothianidin, Flonicamid, Imidacloprid, Nitenpyram, Thiacloprid, Thiamethoxam;

Synthetic coccidiosis compounds, polyetherantibiotics: Amprolium, Robenidin, Toltrazuril, Monensin, Salinomycin, Maduramicin, Lasalocid, Narasin, Semduramicin;

Various: Abamectin (Avermectins), Acequinocyl, Amitraz, Azadirachtin, Bifenazate, Bacillus thuningiensis, Bacillus subtilis, Cartap, Chlorfenapyr, Chlordimeform, Cyromazine, Diafenthiuron, Dinetofuran, Diofenolan, Emamectin, Endosulfan, Epsiprantel, Ethiprole, Fenazaquin, Fipronil, Formetanate, Formetanate hydrochloride, Hydramethylnon, Indoxacarb. 4-{(2Z)-2-({[4-(trifluoro-methoxy)anilino]carbonyl}hydrazono)-2-[3-(trifluoromethyl)-phenyl]ethyl}benzo-nitrile, L-2,3,5,6-tetrahydro-6-phenyl-imidazothiazole, Levamisole, Milbemectin (Milbemycins), Moxidectin, Praziquantel, Pyrantel, Pyridaben, Pymetrozine, Selamectin, Spinosad, Sulfur, Tebufenpyrad, and Thiocyclam.

In general, “parasitically effective amount” means the amount of active ingredient needed to achieve an observable effect on growth, including the effects of necrosis, death, retardation, prevention, and removal, destruction, or otherwise diminishing the occurrence and activity of the target organism. The parasitically effective amount can vary for the various compounds/compositions used in the invention. A parasitically effective amount of the compositions will also vary according to the prevailing conditions such as desired parasiticidal effect and duration, target species, mode of application, and the like.

Examples of Action Against Parasites

1. Screening method to test contact activity against stable fly, yellowfever mosquito, house mosquito, malaria mosquito, cat flea, and brown dog tick via glass contact

Glass vials (20 ml scintillation vials) were treated with 0.5 ml of a solution of active ingredient in acetone. Each vial was rolled uncapped for ca. 10 minutes to allow the a.i. to completely coat the vial and to allow for full drying of the acetone. Insects or ticks were placed into each vial. The vials were kept at 22° C. and were observed for treatment effects at various time intervals. Results are presented in Table 1.

2. Screening method to test contact activity against yellowfever mosquito, southern house mosquito, and malaria mosquito larvae via water treatment

Well plates were used as test arenas. The active ingredient was dissolved in acetone and diluted with water to obtain the concentrations needed. The final solutions containing appr. 1% acetone were placed into each well. Approximately 10 mosquito larvae (4^{th}-instars) in 1 ml water were added to each well. Larvae were fed one drop of liver powder each day. The dishes were covered and maintained at 22° C. Mortality was recorded daily and dead larvae and live or dead pupae were removed daily. At the end of the test remaining live larvae were recorded and percent mortality was calculated. Results are shown in Table 1.

Each test was replicated at least 3 times.

Results

Tests conducted with compounds of formula I-1 and 1-2 showed the following results:

TABLE I
Activity against various species.

| | | | |
|---|---|---|---|
| | | | Days or Hours |
| Pest Common | | | to achieve |
| Name | Pest Latin Name | Rate | 100% mortality |

Screening method to test contact activity via glass contact

| | | | | | |
|---|---|---|---|---|---|
| stable fly | Stomoxys calcitrans | 10 | ppm | 4 | hours |
| yellowfever | Aedes aegypti | 10 | ppm | 4 | hours |
| mosquito | | | | | |
| house | Culex quinquefasciatus | 0.5 | ppm | 4 | hours |
| mosquito | | | | | |
| malaria | Anopheles albimanus | 1 | ppm | 1 | day |
| mosquito | | | | | |
| cat flea | Ctenocephalides felis | 100 | ppm | 2 | days |
| brown | Rhipicephalus | 10 | ppm | 3-5 | days |
| dog tick | sanguineus | | | | |

Screening method to test contact activity via water treatment

| | | | | | |
|---|---|---|---|---|---|
| yellowfever | Aedes aegypti | 10 | ppm | 2 | days |
| mosquito | | | | | |
| house | Culex quinquefasciatus | 10 | ppm | 1 | day |
| mosquito | | | | | |
| malaria | Anopheles albimanus | 1.0 | ppm | 1 | day |
| mosquito | | | | | |

3. Activity against cat flea in an “artificial dog” apparatus

The active ingredient was dissolved in acetone and mixed with an appropriate volume of defibrinated cattle blood. 5 ml of treated blood were poured into a feeding chamber fitted with a paraffin wax film membrane. The chamber with the treated blood was placed over a flea feeding chamber. The test was repeated for each of 5 dose to concentrations of each of the active ingredients. Treatment effects, including knockdown, failure to feed after 24 hours, failure to lay eggs, etc, were observed at various intervals. Control tests were conducted with acetone/blood mixtures.

Results

Tests conducted with 100 ppm of compounds of formula I-1 and 1-2 showed an over 60% killing rate of Ctenocephalides felis.

1-10. (canceled)

11. A method for treating, controlling, preventing or protecting animals against infestation or infection by parasites comprising orally, topically or parenterally administering or applying to the animals a parasitically effective amount of a compound of formula I 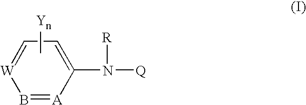
wherein
Q is 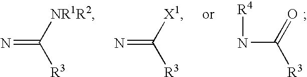
X¹ is chlorine, bromine, or fluorine;
R¹, R² are each independently hydrogen, C₁-C₁₀-alkyl, C₃-C₁₀-alkenyl, C₃-C₁₀-alkynyl, or C₃-C₁₂-cycloalkyl, C₁-C₆-alkylamino, di(C₁-C₆-alkyl)-amino, C₁-C₆-alkylcarbonylamino, C₁-C₆-alkylsulfonyl, or C₁-C₆-alkylsulfinyl, wherein the carbon atoms in these groups may be substituted with
1 to 3 halogen, hydroxy, nitro, cyano, amino, mercapto, C₁-C₆-alkoxy, C₁-C₆-haloalkoxy, C₁-C₆-alkylthio, C₁-C₆-haloalkylthio, C₁-C₆-alkylsulfonyl, C₁-C₆-alkylsulfinyl, C₁-C₆-haloalkylsulfonyl, C₁-C₆-haloalkylsulfinyl, or C₃-C₆-cycloalkyl which may be substituted with 1 to 3 R^{#} groups, or
R^{#} is halogen, cyano, nitro, hydroxy, mercapto, amino, C₁-C₆-alkoxy, C₂-C₆-alkenyloxy, C₂-C₆-alkynyloxy, C₁-C₆-haloalkoxy, C₁-C₆-alkylthio, or C₁-C₆-haloalkylthio, C₁-C₆-alkylsulfonyl, C₁-C₆-alkylsulfinyl, C₁-C₆-alkylamino, di(C₁-C₆alkyl)-amino, C₁-C₆-alkylcarbonyl, C₁-C₆-alkoxycarbonyl, or di(C₁-C₆)-alkylaminocarbonyl;
formyl, C₁-C₆-alkylcarbonyl, C(═O)NR^{a}R^{b}, CO₂R^{c}, R^{d}, R^{e}, phenyl which may be substituted with 1 to 3 R^{#} groups, or pyridyl which may be substituted with 1 to 3 R^{#} groups,
R^{a}, R^{b}, R^{c} are each independently hydrogen or C₁-C₄-alkyl which may be substituted with 1 to 3 groups R^{#};
R^{d} is NRⁱR^{j} or 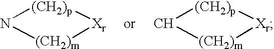
Rⁱ, R^{j} are each independently hydrogen or C₁-C₄-alkyl which may be substituted with 1 to 3 groups R^{#};
p, m are each independently 0, 1, 2, or 3, with the proviso that p and m are not both 0;
X is oxygen, sulfur, amino, C₁-C₄-alkylamino, or phenylamino, or, if p is 0 then X can also be phenoxy or C₁-C₆-alkoxy;
r is 0 or 1;
R^{e} is 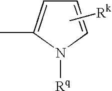
R^{k}, R^{q} are each independently hydrogen or C₁-C₄-alkyl which may be substituted with 1 to 3 groups R^{#}; or
R¹ and R² may be taken together to form a ring represented by the structure 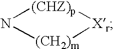
p, m are 1, 2 or 3;
X′ is oxygen, sulfur, amino, C₁-C₄-alkylamino, phenylamino, or methylene;
Z is C₁-C₄-alkyl or phenyl;
R³ is hydrogen, C₁-C₁₀-alkyl, C₂-C₁₀-alkenyl, C₂-C₁₀-alkynyl, C₃-C₁₂-cycloalkyl, wherein the carbon atoms in these groups may be partially or fully halogenated or substituted with
1 to 3 cyano, nitro, hydroxy, mercapto, amino, C₁-C₆-alkyl, C₃-C₆-cycloalkyl, C₁-C₆-alkoxy, C₁-C₆-alkylamino, di(C₁-C₆-alkyl)-amino, C₁-C₆-alkylthio, C₁-C₆-alkylsulfonyl, or C₁-C₆-alkylsulfinyl groups, wherein the carbon atoms in these groups may be substituted by
1 to 3 halogen atoms, a 5- to 6-membered aromatic ring system which may contain 1 to 4 heteroatoms selected from oxygen, sulfur and nitrogen and which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms, or
phenoxy, which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms, or
a 3- to 6-membered saturated or partially unsaturated ring system which contains 1 to 3 heteroatoms selected from oxygen, sulfur and nitrogen and which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms,
a 3- to 6-membered saturated or partially unsaturated ring system which contains 1 to 3 heteroatoms selected from oxygen, sulfur and nitrogen and which is unsubstituted or substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, C₁-C₆-haloalkoxy, nitro, or cyano groups, wherein the carbon atoms in these groups may be substituted by 1 to 3 halogen atoms;
R, R⁴ are each independently hydrogen or C₁-C₆-alkyl, C₁-C₆-alkoxycarbonyl, C₁-C₆-alkylaminocarbonyl, or di(C₁-C₆-alkyl)-aminocarbonyl, wherein the carbon atoms in the these groups may be substituted with 1 to 3 groups R^{#};
A is C—R⁵ or N;
B is C—R⁶ or N;
W is C—R⁷ or N;
with the proviso that one of A, B and W is other than N;
R⁵, R⁶, R⁷ are each independently hydrogen, halogen, nitro, cyano, amino, mercapto, hydroxy, C₁-C₁₀-alkyl, C₂-C₁₀-alkenyl, C₂-C₁₀-alkynyl, C₃-C₆-cycloalkyl, C₁-C₆-alkoxy, C₁-C₆-alkylamino, di(C₁-C₆-alkyl)-amino, C₁-C₆-alkylthio, C₁-C₆-alkylsulfonyl, or C₁-C₆-alkylsulfinyl, wherein the carbon atoms in these groups may be substituted with 1 to 3 groups R^{#}
a 5- to 6-membered aromatic ringsystem which may contain 1 to 4 heteroatoms selected from oxygen, sulfur and nitrogen and which may be substituted with any combination of 1 to 5 halogen atoms, 1 to 3 C₁-C₆-alkyl, C₁-C₆-haloalkyl, C₁-C₆-alkylthio, C₁-C₆-haloalkylthio, C₁-C₆-alkylsulfonyl, C₁-C₆-alkylsulfinyl, C₁-C₆-haloalkylsulfonyl, C₁-C₆-haloalkylsulfinyl, C₁-C₆-alkoxy, C₁-C₆-haloalkoxy, mercapto, hydroxy, amino, nitro, or cyano groups, wherein the carbon atoms in these groups may be substituted with 1 to 3 groups R^{#};
Y is hydrogen, halogen, cyano, nitro, amino, hydroxy, mercapto, C₁-C₆-alkyl, C₂-C₁₀-alkenyl, C₂-C₁₀-alkynyl, C₃-C₆-cycloalkyl, C₁-C₆-alkoxy, C₁-C₆-alkylamino, di(C₁-C₆)-alkylamino, C₁-C₆-alkylthio, C₁-C₆-alkylsulfonyl, or C₁-C₆-alkylsulfinyl, wherein the carbon atoms in these groups may be substituted with 1 to 3 groups R^{#};
n is 0, 1, or 2;
or the enantiomers or diastereomers, veterinarily acceptable salts or esters thereof.

12. The method according to claim 11 wherein the compound of formula I is a compound of formula I-B 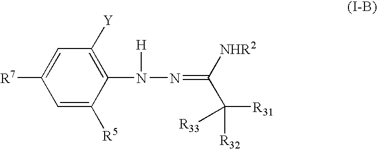
wherein
R⁷ is chlorine or trifluoromethyl;
R⁵ and Y are each independently chlorine or bromine;
R² is C₁-C₆-alkyl, C₃-C₆-alkenyl, C₃-C₆-alkynyl, or
C₃-C₆-cycloalkyl which may be substituted with 1 to 3 halogen atoms, or
C₂-C₄-alkyl which is substituted by C₁-C₄-alkoxy;
R³¹ and R³² are C₁-C₆-alkyl or may be taken together to form C₃-C₆-cycloalkyl which may be unsubstituted or substituted by 1 to 3 halogen atoms;
R³³ is hydrogen or C₁-C₆-alkyl,
or the enantiomers or veterinarily acceptable salts thereof.

13. The method according to claim 11 wherein the compound of formula I is a compound of formula I-1

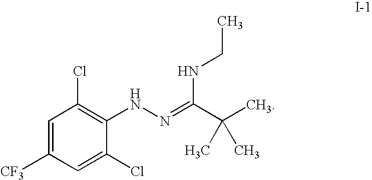

14. The method according to claim 11 wherein the compound of formula I is a compound of formula I-2

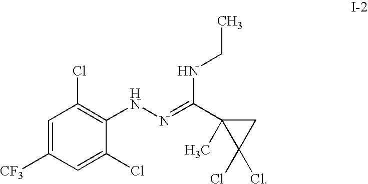

15. The method according to claim 11 wherein the parasites are selected from the Diptera, Siphonaptera, and Ixodida orders.

16. The method according to claim 12 wherein the parasites are selected from the Diptera, Siphonaptera, and Ixodida orders.

17. The method according to claim 13 wherein the parasites are selected from the Diptera, Siphonaptera, and Ixodida orders.

18. The method according to claim 14 wherein the parasites are selected from the Diptera, Siphonaptera, and Ixodida orders.

19. The method according to claim 11 wherein the animals are cats or dogs.

20. The method according to claim 12 wherein the animals are cats or dogs.

21. The method according to claim 13 wherein the animals are cats or dogs.

22. The method according to claim 14 wherein the animals are cats or dogs.

23. The method according to claim 15 wherein the animals are cats or dogs.

24. A process for the preparation of a composition for treating, controlling, preventing or protecting animals against infestation or infection by parasites, the process comprising:
providing a parasitically effective amount of a compound of formula I: 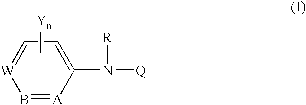
wherein
Q is 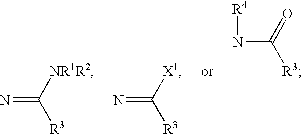
X¹ is chlorine, bromine, or fluorine;
R¹, R² are each independently hydrogen, C₁-C₁₀-alkyl, C₃-C₁₀-alkenyl, C₃-C₁₀-alkynyl, or C₃-C₁₂-cycloalkyl, C₁-C₆-alkylamino, di(C₁-C₆-alkyl)-amino, C₁-C₆-alkylcarbonylamino, C₁-C₆-alkylsulfonyl, or C₁-C₆-alkylsulfinyl, wherein the carbon atoms in these groups may be substituted with
1 to 3 halogen, hydroxy, nitro, cyano, amino, mercapto, C₁-C₆-alkoxy, C₁-C₆-haloalkoxy, C₁-C₆-alkylthio, C₁-C₆-haloalkylthio, C₁-C₆-alkylsulfonyl, C₁-C₆-alkylsulfinyl, C₁-C₆-haloalkylsulfonyl, C₁-C₆-haloalkylsulfinyl, or C₃-C₆-cycloalkyl which may be substituted with 1 to 3 R^{#} groups, or
R^{#} is halogen, cyano, nitro, hydroxy, mercapto, amino, C₁-C₆-alkoxy, C₂-C₆-alkenyloxy, C₂-C₆-alkynyloxy, C₁-C₆-haloalkoxy, C₁-C₆-alkylthio, or C₁-C₆-haloalkylthio, C₁-C₆-alkylsulfonyl, C₁-C₆-alkylsulfinyl, C₁-C₆-alkylamino, di(C₁-C₆alkyl)-amino, C₁-C₆-alkylcarbonyl, C₁-C₆-alkoxycarbonyl, or di(C₁-C₆)-alkylaminocarbonyl;
formyl, C₁-C₆-alkylcarbonyl, C(═O)NR^{a}R^{b}, CO₂R^{c}, R^{d}, R^{e}, phenyl which may be substituted with 1 to 3 R^{#} groups, or pyridyl which may be substituted with 1 to 3 R^{#} groups,
R^{a}, R^{b}, R^{c} are each independently hydrogen or C₁-C₄-alkyl which may be substituted with 1 to 3 groups R^{#};
R^{d} is NRⁱR^{j} or 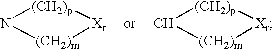
Rⁱ, R^{j} are each independently hydrogen or C₁-C₄-alkyl which may be substituted with 1 to 3 groups R^{#};
p, m are each independently 0, 1, 2, or 3, with the proviso that p and m are not both 0;
X is oxygen, sulfur, amino, C₁-C₄-alkylamino, or phenylamino, or, if p is 0 then X can also be phenoxy or C₁-C₆-alkoxy;
r is 0 or 1;
R^{e} is 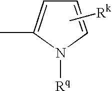
R^{k}, R^{q} are each independently hydrogen or C₁-C₄-alkyl which may be substituted with 1 to 3 groups R^{#}; or
R¹ and R² may be taken together to form a ring represented by the structure 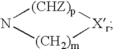
p, mare 1, 2 or 3;
X′ is oxygen, sulfur, amino, C₁-C₄-alkylamino, phenylamino, or methylene;
Z is C₁-C₄-alkyl or phenyl;
R³ is hydrogen, C₁-C₁₀-alkyl, C₂-C₁₀-alkenyl, C₂-C₁₀-alkynyl, C₃-C₁₂-cycloalkyl, wherein the carbon atoms in these groups may be partially or fully halogenated or substituted with
1 to 3 cyano, nitro, hydroxy, mercapto, amino, C₁-C₆-alkyl, C₃-C₆-cycloalkyl, C₁-C₆-alkoxy, C₁-C₆-alkylamino, di(C₁-C₆-alkyl)-amino, C₁-C₆-alkylthio, C₁-C₆-alkylsulfonyl, or C₁-C₆-alkylsulfinyl groups, wherein the carbon atoms in these groups may be substituted by
1 to 3 halogen atoms, a 5- to 6-membered aromatic ring system which may contain 1 to 4 heteroatoms selected from oxygen, sulfur and nitrogen and which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms, or
phenoxy, which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms, or
a 3- to 6-membered saturated or partially unsaturated ring system which contains 1 to 3 heteroatoms selected from oxygen, sulfur and nitrogen and which may be substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, nitro, or cyano groups, wherein the carbon atoms in these groups may be substituted by 1 to 3 halogen atoms,
a 3- to 6-membered saturated or partially unsaturated ring system which contains 1 to 3 heteroatoms selected from oxygen, sulfur and nitrogen and which is unsubstituted or substituted with any combination of 1 to 5 halogen atoms, 1 to 3 C₁-C₆-alkyl, C₁-C₆-alkylthio, C₁-C₆-alkylsulfonyl, C₁-C₆-alkylsulfinyl, C₁-C₆-alkoxy, C₁-C₆-haloalkoxy, nitro, or cyano groups, wherein the carbon atoms in these groups may be substituted by 1 to 3 halogen atoms;
R, R⁴ are each independently hydrogen or C₁-C₆-alkyl, C₁-C₆-alkoxycarbonyl, C₁-C₆-alkylaminocarbonyl, or di(C₁-C₆-alkyl)-aminocarbonyl, wherein the carbon atoms in the these groups may be substituted with 1 to 3 groups R⁴;
A is C—R⁵ or N;
B is C—R⁶ or N;
W is C—R⁷ or N;
with the proviso that one of A, B and W is other than N;
R⁵, R⁶, R⁷ are each independently hydrogen, halogen, nitro, cyano, amino, mercapto, hydroxy, C₁-C₁₀-alkyl, C₂-C₁₀-alkenyl, C₂-C₁₀-alkynyl, C₃-C₆-cycloalkyl, C₁-C₆-alkoxy, C₁-C₆-alkylamino, di(C₁-C₆-alkyl)-amino, C₁-C₆-alkylthio, C₁-C₆-alkylsulfonyl, or C₁-C₆-alkylsulfinyl, wherein the carbon atoms in these groups may be substituted with 1 to 3 groups R^{#}
a 5- to 6-membered aromatic ringsystem which may contain 1 to 4 heteroatoms selected from oxygen, sulfur and nitrogen and which may be substituted with any combination of 1 to 5 halogen atoms, 1 to 3 C₁-C₆-alkyl, C₁-C₆-haloalkyl, C₁-C₆-alkylthio, C₁-C₆-haloalkylthio, C₁-C₆-alkylsulfonyl, C₁-C₆-alkylsulfinyl, C₁-C₆-haloalkylsulfonyl, C₁-C₆-haloalkylsulfinyl, C₁-C₆-alkoxy, C₁-C₆-haloalkoxy, mercapto, hydroxy, amino, nitro, or cyano groups, wherein the carbon atoms in these groups may be substituted with 1 to 3 groups R^{#};
Y is hydrogen, halogen, cyano, nitro, amino, hydroxy, mercapto, C₁-C₆-alkyl, C₂-C₁₀-alkenyl, C₂-C₁₀-alkynyl, C₃-C₆-cycloalkyl, C₁-C₆-alkoxy, C₁-C₆-alkylamino, di(C₁-C₆)-alkylamino, C₁-C₆-alkylthio, C₁-C₆-alkylsulfonyl, or C₁-C₆-alkylsulfinyl, wherein the carbon atoms in these groups may be substituted with 1 to 3 groups R^{#};
n is 0, 1, or 2;
or an enantiomer or diastereomer, veterinarily acceptable salt or ester thereof; and
dissolving the compound of formula I in a physiologically tolerable carrier.

25. The process according to claim 24, wherein the physiologically tolerable carrier is a solvent comprising water, ethanol, butanol, benzyl alcohol, glycerol, propylene glycol, polyethylene glycol, N-methyl-pyrrolidone, 2-pyrrolidone, polypropylene glycol, phenyl ethanol, phenoxy ethanol, ethyl acetate, butyl acetate, benzyl benzoate, dipropyleneglycol monomethylether, acetone, methylethylketone, aromatic hydrocarbons, vegetable oils, synthetic oils, dimethylformamide, dimethylacetamide, transcutol, solketal, propylenecarbonate or mixtures thereof.

26. The process according to claim 24, wherein the physiologically tolerable carrier is an oil comprising a vegetable oil or synthetic oil suitable for injection.

27. The process according to claim 24, further comprising adding a thickener to the physiologically tolerable carrier.

28. The process according to claim 27, wherein the thickener is bentonite, colloidal silicic acid, aluminium monostearate, a cellulose derivative, a polyvinyl alcohol or a copolymer of a polyvinyl alcohol, an acrylate or a methacrylate.

29. The process according to claim 27, wherein the composition is a gel.

30. The process according to claim 24, wherein the carrier comprises a hydrophobic phase, a hydrophilic phase and an emulsifier such that the compound of formula I and the carrier forms an emulsion.

31. The process according to claim 30, wherein the compound of formula I is dissolved in one of the hydrophobic phase or the hydrophilic phase, and is homogenized with the emulsifier and the other phase.

32. The process according to claim 24, wherein the physiologically tolerable carrier comprises one or more physiologically tolerable solid inert substances.

33. The process according to claim 32, wherein the solid inert substance is selected from the group consisting of sodium chloride, calcium carbonate, hydrogencarbonates, aluminium oxides, titanium oxide, silicic acids, argillaceous earths, precipated or colloidal silica, phosphates, sugar, cellulose, milk powder, animal meal, grain meals and starches.

34. The process according to claim 24 wherein the compound of formula I is a compound of formula I-B 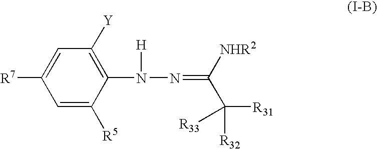
wherein
R⁷ is chlorine or trifluoromethyl;
R⁵ and Y are each independently chlorine or bromine;
R² is C₁-C₆-alkyl, C₃-C₆-alkenyl, C₃-C₆-alkynyl, or
C₃-C₆-cycloalkyl which may be substituted with 1 to 3 halogen atoms, or
C₂-C₄-alkyl which is substituted by C₁-C₄-alkoxy;
R³¹ and R³² are C₁-C₆-alkyl or may be taken together to form C₃-C₆-cycloalkyl which may be unsubstituted or substituted by 1 to 3 halogen atoms;
R³³ is hydrogen or C₁-C₆-alkyl,
or the enantiomers or veterinarily acceptable salts thereof.

35. The process according to claim 24 wherein the compound of formula I is a compound of formula I-1

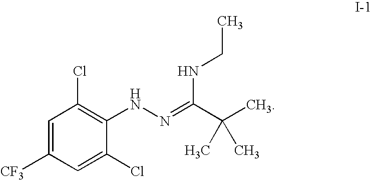

36. The process according to claim 24 wherein the compound of formula I is a compound of formula I-2 [chemical structure]
